SEQUENCE LISTING (20 sequences)

SEQ ID NO: 1 - DNA, 60 bp, Equus caballus
ctccctccacagcatcagaccgggaaattaaacccctctgggctcgtggagcggagtgag

SEQ ID NO: 2 - DNA, 60 bp, Equus caballus
atcaggcaccaaagtccaccaagggcagctcttggtcgtgtggatgtgagcaattagttt

SEQ ID NO: 3 - DNA, 60 bp, Equus caballus
atgtgaaggatgctttgtggataatgataaatttagagatgctttccagttctgtgatgc

SEQ ID NO: 4 - DNA, 60 bp, Equus caballus
gctcaatgaagctcaagcccaggaaatatgaagagaactaccacaaaataaaatcaagtg

SEQ ID NO: 5 - DNA, 60 bp, Equus caballus
gctgggagggagggattgagaggactgtgctatgaagtatctgcattacacacgaagctt

SEQ ID NO: 6 - DNA, 60 bp, Equus caballus
caattgtcaggaggagaagacgttgaacacgtaactgcaaaaatatttgaccatcaaaag

SEQ ID NO: 7 - DNA, 60 bp, Equus caballus
tccacttttgtgcgaaatctcccgctttgagagaaaagtcaagatgaggggctctgctac

SEQ ID NO: 8 - DNA, 60 bp, Equus caballus
atatatatgccatctaattttggtttatgcttaccacatttttcaattttattgaattaa

SEQ ID NO: 9 - DNA, 60 bp, Equus caballus
tctgaagcagtggatttcctcttccctggctggcttaatcggaatttcccatccccaagc

SEQ ID NO: 10 - DNA, 60 bp, Equus caballus
gaccagatcccaggctggatctgggcatcagtggccagctgtggatcacgaaattttcaa

SEQ ID NO: 11 - DNA, 60 bp, Equus caballus
aaccacatgaaaatagctctggtcaccaaaagaacaccaaaaggaacgctaaaacacaag

SEQ ID NO: 12 - DNA, 60 bp, Equus caballus
atggctggggcctccttcactgcaggacagagagggggccaccctgagtctctgccggtg

SEQ ID NO: 13 - DNA, 60 bp, Equus caballus
aaactatctagcaccctcaacaaggtaaaatctgtaattccaatttaaaaagcatgaaaa

SEQ ID NO: 14 - DNA, 60 bp, Equus caballus
tgtaatgtccatttcctcccccacagacactgggctgatgacctgcacagtggatgtttg

SEQ ID NO: 15 - DNA, 60 bp, Equus caballus
gtgccggttggtggaagtgattcattacatccagtccacaccgaaggggaggggaattag

SEQ ID NO: 16 - DNA, 60 bp, Equus caballus
tttggatctgtgaggtacctcactacaataggtatgagatattcagagaacatcagtaga

SEQ ID NO: 17 - DNA, 30 bp, Artificial Sequence
  Forward PCR primer
ggaagtttctaaacatttttgaaggctttt

SEQ ID NO: 18 - DNA, 23 bp, Artificial Sequence
  Reverse PCR primer
ggagggaagtcaattgacaaacg

SEQ ID NO: 19 - DNA, 14 bp, Artificial Sequence
  Mutant probe (FAM)
cctccacggcatca

SEQ ID NO: 20 - DNA, 16 bp, Artificial Sequence
  Reference probe (VIC)
tccctccacagcatca

(19) United States
(12) Patent Application Publication
(10) Pub. No.: US 2024/0043926 A1
(43) Pub. Date: Feb. 8, 2024

(54) BIOMARKERS FOR CONFORMATION OF RIDING TRAITS IN HORSES

(71) Applicants: Gabriella LINDGREN, Knivsta (SE); Maria ROSENGREN, Uppsala (SE); Susanne ERIKSSON, Vänge (SE); THE REGENTS OF THE UNIVERSITY OF CALIFORNIA, Oakland, CA (US)

(72) Inventors: Gabriella LINDGREN, Knivsta (SE); Maria ROSENGREN, Uppsala (SE); Susanne ERIKSSON, Vänge (SE); Marina SOLÉ BERGA,, Mollerussa (ES); Rebecca BELLONE, Woodland, CA (US)

(21) Appl. No.: 18/551,126

(22) PCT Filed: Mar. 18, 2022
(86) PCT No.: PCT/SE2022/050259

§ 371 Date: Sep. 18, 2023

Related U.S. Application Data
(60) Provisional application No. 63/163,398, filed on Mar. 19, 2021.

(30) Foreign Application Priority Data
Apr. 9, 2021 (SE) 2150444-4

(51) Int. Cl. C12Q 1/6876

(52) U.S. Cl. CPC C12Q 1/6876; C12Q 2600/124; C12Q 2600/156

(57) ABSTRACT

The invention relates to a method for determining, in a sample comprising nucleic acid molecules obtained from the horse, presence or absence of at least one biomarker useful in predicting conformation of back and croup and/or gait quality and/or gait type trot or pace of the horse. The at least one biomarker is located in a region of from nucleotide position 44,000,000 to nucleotide position 47,000,000 on Equus caballus chromosome 22 (ECA22).

### TECHNICAL FIELD

The present invention generally relates to biomarkers for conformation of back and croup in horses, and in particular to such biomarkers that influences gait quality and/or performance type and uses thereof.

### BACKGROUND

Associations of body measurements with locomotor health and sports performance have been reported in many different horse breeds, including Icelandic horses. Discriminant analyses have shown that several morphological features distinguish with high accuracy between low-class and high-class Icelandic horses with respect to different riding ability traits. The most important features for gait ability in Icelandic horses are the height of the horse at front compared to hind (uphill conformation) with well-balanced backline, croup proportions and width of chest. The analyses also indicated the disadvantage of a forward inclination in the back or a sway back. Conformation of the back and croup, thus, plays a major role on riding ability in Icelandic horses.

The Icelandic horse official breeding goal promotes five-gaited horses with a functional and aesthetically pleasing conformation. Zoometric measurements and subjective scores for conformation and riding ability traits are recorded at breeding field tests. Genetic correlations between conformation of back and croup, and gait qualities have been estimated to be moderate to high (0.19-0.54). Furthermore, moderate heritabilities (0.29-0.31) have been estimated for the subjectively scored back and croup trait and the objectively measured zoometric traits pertaining to conformation of back and croup (0.20-0.25). For the subjectively scored riding ability traits, the heritability estimates range from 0.18 (walk) to 0.60 (pace).

Despite conformation traits being moderately heritable in the Icelandic horse, only mutations in the Myostatin gene have previously been associated with conformation traits, i.e., estimated breeding values of neck, withers and shoulders. In other horse breeds, as well as other species, many different genes have been shown to influence body size. Ligand dependent nuclear receptor corepressor like (LCORL), non-SMC condensin I complex subunit G (NCAPG) and high mobility group AT-hook (HMGA2) are major genes known to regulate body size in mammals including humans, cattle, sheep, dogs and horses. These genes, along with other genes, such as Zinc finger and AT-hook domain containing (ZFAT) and LIM and SH3 protein 1 (LASP1), affect not only the body size of the horse but more specifically the height at withers. Three novel missense variants located in the ADAM metallopeptidase with thrombospondin type 1 motif (ADAMTS17), osteocrin (OSTN) and growth hormone 1 (GH1) genes explained 61% of the variance of withers height in Shetland pony-related breeds. Other additional quantitative trait loci have also shown significant associations with morphometric angular measurements, with regions on chromosomes Equus caballus chromosome 28 (ECA28) and ECA29 associated with poll angle in horses. However, the genes behind many other conformation traits are still unknown.

Genetic tests for predicting the pattern of locomotion in a horse including its gaits and athletic performance are known in the art, see for instance, U.S. Pat. No. 8,771,943; US 2019/0382843; WO 2019/204340.

There is still a need for genetic tests for predicting conformation of back and croup of horses and for predicting gait type and quality of horses.

### SUMMARY

It is a general objective to provide biomarkers for predicting conformation of back and croup of horses.

It is a particular objective to provide biomarkers for predicting gait quality, in particular lateral gait quality, of horses.

It is another particular objective to provide biomarkers for predicting gait performance type, in particular pace or trot, of horses.

These and other objectives are met by embodiments as disclosed herein.

The present invention is defined in the independent claims. Further embodiments of the invention are defined in the dependent claims.

An aspect of the invention relates to a method for predicting conformation of back and croup and/or gait quality and/or gait performance type trot or pace of a horse. The method comprises determining, in a sample comprising nucleic acid molecules obtained from the horse, presence or absence of at least one biomarker useful in predicting conformation of back and croup and/or gait quality and/or gait performance type trot or pace of the horse. The at least one biomarker is located in a region of from nucleotide position 44,000,000 to nucleotide position 47,000,000 on Equus caballus chromosome 22 (ECA22).

Another aspect of the invention relates to a method for selection a horse for breeding. The method comprises determining, in a sample comprising nucleic acid molecules obtained from the horse, the allele of at least one biomarker useful in predicting conformation of back and croup and/or gait quality and/or gait performance type trot or pace of the horse. The at least one biomarker is located in a region of from nucleotide position 44,000,000 to nucleotide position 47,000,000 on ECA22. The method also comprises selecting the horse for breeding based on the determined allele of the at least one biomarker.

A further aspect of the invention relates to a method for selecting a training scheme for a horse. The method comprises determining, in a sample comprising nucleic acid molecules obtained from the horse, the allele of at least one biomarker useful in predicting conformation of back and croup and/or gait quality and/or gait performance type trot or pace of the horse. The at least one biomarker is located in a region of from nucleotide position 44,000,000 to nucleotide position 47,000,00 on ECA22. The method also comprises selecting the training scheme for the horse based on the determined allele of the at least one biomarker.

Yet another aspect of the invention relates to a kit for predicting conformation of back and croup and/or gait quality and/or gait performance type trot or pace of a horse. The kit comprises at least one oligonucleotide probe capable of forming a hybridized nucleic acid with a single nucleotide polymorphism (SNP) or a nucleic acid region flanking the SNP. The SNP is selected from the group consisting of a SNP located at position 45,363,022 on ECA22, a SNP located at position 45,388,495 on ECA22, a SNP located at position 45,445,814 on ECA22, a SNP located at position 45,471,357 on ECA22, a SNP located at position 45,494,455 on ECA22, a SNP located at position 45,500,367 on ECA22, a SNP located at position 45,524,597 on ECA22, a SNP located at position 45,532,931 on ECA22, a SNP located at position 45,622,744 on ECA22, and a SNP located at position 45,662,708. The kit also comprises instructions for predicting conformation of back and croup and/or gait quality and/or gait performance type trot or pace of the horse based on the horse's genotype at the SNP.

A further aspect of the invention relates to a kit for predicting conformation of back and croup and/or gait quality and/or gait performance type trot or pace of a horse. The kit comprises at least one oligonucleotide probe capable of forming a hybridized nucleic acid with a SNP or a nucleic acid region flanking the SNP. The SNP is selected from the group consisting of a SNP located at position 45,616,738 on ECA22, a SNP located at position 45,622,744 on ECA22, and a SNP located at position 45,662,708 on ECA22. The kit also comprises instructions for predicting conformation of back and croup and/or gait quality and/or gait performance type in the horse based on the horse's genotype at the SNP.

The inventors have identified a quantitative trait locus (QTL) for the conformation of back and croup of horses. The QTL was also determined to be of importance for the quality of gaits, in particular lateral gaits but also gait performance type, e.g., trot or pace. Horses with a favorable haplotype were more inclined to have a well-balanced backline with an uphill conformation and had higher scores for the tölt and pace.

### BRIEF DESCRIPTION OF THE DRAWINGS

The embodiments, together with further objects and advantages thereof, may best be understood by making reference to the following description taken together with the accompanying drawings, in which:

FIG. 1. Genome-wide association (GWA) results for the score of back and croup. (A) Quantile-quantile (QQ) plot where the lines marked with arrows represent the 0.05-0.95 confidence interval. The estimated lambda value was 0.98 (SE 2.55×10⁻⁵). (B) Manhattan plot from the mixed model association analysis. The top horizontal line indicates Bonferroni significance threshold (p<6.9×10⁻⁸) and the bottom horizontal line indicates the suggestive genome-wide significance level (p<1.0×10⁻⁵). (C) Linkage disequilibrium (LD) Manhattan plot on ECA22 with the top SNP as an open circle. Thirteen SNPs reached the suggestive threshold of which ten were in LD (marked with arrows). All positions refer to EquCab3.0.

FIG. 2. Examples of Icelandic horses representing high and low score of back and croup. (A) Icelandic horse that represents high score of back and croup. The backline is well-balanced and the back is wide and well-muscled. (Photo: Hrefna Maria Ómarsdóttir). (B) Icelandic horse that represents a low score of back and croup with a forward sloping backline and a less muscled croup. (Photo: The Swedish Icelandic Horse Association, SIF).

FIG. 3. Distribution of scores for back and croup in 177 horses.

FIG. 4. Zoometric measurements recorded at standardized breeding field tests for Icelandic horses (1).

FIG. 5. Multidimensional scaling (MDS) plot for the score back and croup. Visualization of population stratification across 177 Icelandic horses that passed the quality control (QC) for the score back and croup. Dots represent horses that had a score lower than the mean 8.1 and triangles represent horses that had a score higher or equal to 8.1.

### DETAILED DESCRIPTION

The present invention generally relates to biomarkers for conformation of back and croup in horses, and in particular to such biomarkers that influences gait quality and/or gait performance type and uses thereof.

The back and croup is a complex trait in horses, with muscular as well as skeletal features of both the back and the croup subjectively assessed and scored together as a single trait. The present invention is based on the detection of a novel quantitative trait locus (QTL) associated with back and croup conformation and that influences various riding ability and conformation traits, including gait quality and performance and in particular lateral gait quality and gait performance type, e.g., trot or pace.

An aspect of the invention relates to a method for predicting conformation of back and croup and/or gait quality and/or gait performance type trot or pace of a horse. The method comprises determining, in a sample comprising nucleic acid molecules obtained from the horse, presence or absence of at least one biomarker useful in predicting conformation of back and croup and/or gait quality and/or gait performance type trot or pace of the horse. According to the invention, the at least one biomarker is located in a region of from nucleotide position 44,000,000 to nucleotide position 47,000,000 on Equus caballus chromosome 22 (ECA22).

The method optionally comprises predicting conformation of back and croup and/or gait quality and/or gait performance type trot or pace of the horse based on the determined presence or absence of at least one biomarker.

The conformation traits back and croup are traditionally assessed at breeding field tests and are subjectively scored on a scale from 5 to 10 with 0.5 intervals, where a score of 5 is only given if a trait is not presented. Assessment of the trait back and croup comprises several aspects of the conformation of the back, croup and loins. The slope and shape of the backline, which is defined as the line from the base of withers to the lumbosacral joint, are assessed. Length and slope of the croup are also assessed, as well as the width and muscularity of the back, the length and width of the loins and the form and muscularity of the croup (33). A high score for back and croup represents a strong, well-balanced backline and a well-muscled wide back. The croup should be long, evenly formed, well-muscled and adequately sloping. A low score is associated with a swayback, stiff or forward sloping backline, a too short or too long and/or unevenly formed croup and poorly muscled back and croup (33). When the judging panel has reached a consensus on a score for back and croup according to the judging scale, they have the possibility to use standardized marks to describe the most prominent positive and/or negative attributes of the trait.

The conformation of back and croup is of importance for the gaits of horses. In particular, experimental data as presented herein show that the at least one biomarker is useful in predicting conformation of back and croup of the horse and riding ability traits of the horse. In more detail, the at least one biomarker was determined to be useful in predicting gait quality and performance. In more detail, the at least one biomarker is useful in predicting lateral gait quality of the horse, and preferably gait quality of tölt and/or pace. The at least one biomarker is also useful in predicting gait performance type of the horse, preferably predicting whether the horse is a trotter or a pacer.

Gait as used herein is the pattern of the legs of horses during locomotion over a solid substrate. Traditionally horse gaits have been divided into natural gaits, including walk, trot, canter, gallop and back, and artificial or ambling gaits, including running walk, slow gait, pace, rack, tölt and paso gaits. Lateral gaits include running walk, slow gait, pace, rack, tölt and paso gaits.

Generally, features of gaits, such as beat, suppleness, stride length, leg-action, speed capacity, collection and lightness, are taken into account when assessing the quality of the gaits (33).

Hence, the present invention also relates to a method for predicting gait quality, preferably lateral gait quality and more preferably lateral gait quality of pace and/or tölt, of a horse. The method comprises determining, in a sample comprising nucleic acid molecules obtained from the horse, presence or absence of at least one biomarker useful in predicting gait quality, preferably lateral gait quality and more preferably lateral gait quality of pace and/or tölt, of the horse. According to the invention, the at least one biomarker is located in a region of from nucleotide position 44,000,000 to nucleotide position 47,000,000 on ECA22.

The method optionally comprises predicting gait quality, preferably lateral gait quality and more preferably lateral gait quality of pace and/or tölt, of the horse based on the based on the determined presence or absence of at least one biomarker.

Trot is a two-beat gait, in which the horse moves its legs in unison in diagonal pairs. Pace is a lateral two-beat gait, in which the two legs on the same side of the horse move forward together.

The present invention also relates to a method for predicting gait performance type of a horse. The method comprises determining, in a sample comprising nucleic acid molecules obtained from the horse, presence or absence of at least one biomarker useful in predicting gait performance type of the horse, preferably predicting the horse to be a trotter or pacer. According to the invention, the at least one biomarker is located in a region of from nucleotide position 44,000,000 to nucleotide position 47,000,000 on ECA22.

The method optionally comprises predicting gait performance type, preferably trot or pace, of the horse based on the determined presence or absence of at least one biomarker.

The term “nucleotide position” as used herein relates to the NCBI Equus caballus Annotation Release 103 having assembly name EquCab3.0 and RefSeq assembly accession no GCF_002863925.1 and GenBank assembly accession no. GCA_002863925.1 (https://www.ncbi.nlm.nih.gov/assembly/GCF_002863925.1/). Equus caballus chromosome 22 (ECA22) has GenBank accession number CM009169 and version 1 (CM009169.1) and NCBI reference sequence accession number NC_009165 and version 3 (NC_009165.3).

The sample is a biological sample that comprises nucleic acid molecules and is obtained from the horse. The nucleic acid molecules could be present as free nucleic acid molecules in the sample. Alternatively, the sample could contain nucleated cells from the horse and where these nucleated cells contain a nucleus comprising the nucleic acid molecules, and in particular a copy of the genome of the horse. The sample could be a body fluid sample comprising nucleic acid molecules, such as a body fluid sample comprising nucleated cells. Illustrative, but non-limiting, examples of such body fluid samples include a blood sample, a saliva sample, a breast milk sample, a vaginal lubrication sample, and a semen sample. Alternatively, the sample could be a body tissue sample including, but not limited to, a hair sample, a hair root sample or a biopsy sample.

The nucleic acid molecules could be deoxyribonucleic acid (DNA) molecules or ribonucleic acid (RNA) molecules, including complementary DNA (cDNA) molecules. In a particular embodiment, the nucleic acid molecules are DNA molecules and preferably genomic DNA.

Nucleic acid molecules can extracted, isolated and optionally purified from the sample according to well-known nucleic acid extraction, isolation and purification methods. For instance, standard protocol for the isolation of genomic DNA could be used, such as are inter alia referred to in (47, 48).

The term “biomarker” is generally defined herein as a biological indicator, such as a particular molecular feature, that may affect or be related to predicting at least one trait of a horse. For example, in certain embodiments of the present invention, the biomarker is a genetic marker, such as a single nucleotide polymorphism (SNP), e.g., a particular genotype at a SNP.

The novel QTL of the invention is present in a portion of ECA22 harboring the genes Chromosome 22 C20orf85 homolog (C22H20orf85), Ankyrin repeat domain 60 (ANKRD60) and LOC100056167. The genes C22H20orf85 and ANKRD60 are linked to scoliosis traits in humans and in particular to adolescent idiopathic scoliosis (AIS). The gene ANKRD60 is also associated with body height in humans. The gene LOC100056167 is described as serine/threonine-protein phosphatase 4 regulatory subunit 1 and is not well annotated in horses but has 84.17% sequence identity with the pseudogene JP4R1L in humans. PPP4R1L has a potential effect on bone mineral density as it has a protein phosphatase regulator activity. PPP4R1L is regulated by an enhancer with potential implications on body height and BMI-adjusted waist circumference in humans.

In an embodiment, the at least one biomarker is located in a region of from nucleotide position 44,347,522 to nucleotide position 46,662,708 on ECA22.

In a particular embodiment, the at least one biomarker is located in a region of from nucleotide position 45,347,522 to nucleotide position 46,662,708 on ECA22.

In another particular embodiment, the at least one biomarker is located in a region of from nucleotide position 44,347,522 to nucleotide position 45,662,708 on ECA22.

In a preferred embodiment, the at least one biomarker is located in a region of from nucleotide position 45,347,522 to nucleotide position 45,662,708 on ECA22.

As is further disclosed herein 383,896 single nucleotide polymorphisms (SNPs) and 177 horses passed quality control (QC) and were included in a genome-wide association (GWA) analysis. Sixteen of the 50 top SNPs were located on ECA22: 45,347,522-45,662,708, see Table 1.

TABLE 1
Summary of the SNPs on ECA22 from the GWA analysis for score of back and croup

| | | | | | | | Number | | | |
|---|---|---|---|---|---|---|---|---|---|---|
| SNP Probe Set | | Position | | | Reference | Alternate | of | | SE of | Raw p- |
| ID | ECA | (bp) | A1 | A2 | Allele | Allele | horses | effB | effB | value |
| AX-102949172 | 22 | 45,532,931 | G | A | A | G | 177 | −0.32 | 0.06 | 2.67E−07 |
| AX-104791028 | 22 | 45,622,744 | G | A | A | G | 177 | −0.31 | 0.06 | 6.00E−07 |
| AX-103174786 | 22 | 45,494,455 | G | T | T | G | 177 | −0.30 | 0.06 | 9.66E−07 |
| AX-103610371 | 22 | 45,471,357 | C | A | A | C | 176 | −0.31 | 0.06 | 1.01E−06 |
| AX-103572410 | 22 | 45,524,597 | G | T | T | G | 177 | −0.30 | 0.06 | 1.21E−06 |
| AX-104515875 | 22 | 45,363,022 | G | A | G | A | 177 | 0.29 | 0.06 | 1.83E−06 |
| AX-103805460 | 22 | 45,388,495 | C | T | T | C | 177 | −0.28 | 0.06 | 2.84E−06 |
| AX-104362627 | 22 | 45,497,961 | C | T | T | C | 177 | −0.28 | 0.06 | 3.45E−06 |
| AX-103795824 | 22 | 45,451,809 | G | A | A | G | 176 | −0.28 | 0.06 | 7.65E−06 |
| AX-103100016 | 22 | 45,500,367 | G | A | A | G | 177 | −0.28 | 0.06 | 8.06E−06 |
| AX-103683359 | 22 | 45,662,708 | C | T | T | C | 177 | −0.28 | 0.06 | 9.06E−06 |
| AX-103453843 | 22 | 45,347,522 | T | C | T | C | 177 | −0.27 | 0.06 | 9.09E−06 |
| AX-104157798 | 22 | 45,445,814 | T | C | C | T | 171 | −0.28 | 0.06 | 1.11E−05 |
| AX-103739323 | 22 | 45,539,096 | A | G | G | A | 177 | −0.27 | 0.06 | 1.30E−05 |
| AX-103902715 | 22 | 45,396,929 | G | A | A | G | 176 | −0.25 | 0.06 | 3.44E−05 |
| AX-104277754 | 22 | 45,653,638 | G | A | A | G | 176 | −0.24 | 0.06 | 8.54E−05 |

SNP Probe Set ID: ID of SNP from Axiom ™ Equine Genotyping Array annotation file;
ECA: Equus caballus chromosome;
A1: Major allele;
A2: Minor allele;
effB: Estimated allele substitution effect;
SE of effB: Standard error of the estimated allele substitution effect

“Single nucleotide polymorphism” or “SNP” as used herein refers to a single nucleotide polymorphism at a particular position in the horse genome that varies among a population of individuals. A SNP can be identified by its location within ECA22, i.e., nucleotide position on ECA22, or by its name as shown in Tables 1 to 4. SNPs identified as being useful for predicting conformation of the back and croup and gait quality are shown in Tables 1 to 4. For example, the SNP AX-102949172 in Tables 1 to 4 indicates that the nucleotide base (or the allele) at nucleotide position 45,532,931 on ECA22 may be either guanine (G) or adenine (A). The allele associated with or indicative for conformation of the back and croup and for the gait quality of a horse is in such an example SNP AX-102949172 of Table 1 to 4 guanine (G).

In an embodiment, determining presence or absence of at least one biomarker comprises determining genotype of at least one SNP selected from the group consisting of the SNPs listed in Table 1.

Thus, the SNPs listed in Table 1 are indicative of the conformation of the back and croup and for the gait quality of a horse and may thereby be used as biomarker or genetic marker in predicting the conformation of the back and croup and/or for the gait quality of the horse.

In a particular embodiment, determining genotype of at least one SNP selected from the group consisting of the SNPs listed in Table 1 comprises determining whether the at least one SNP has the reference allele in Table 1 or at he alternate allele in Table 1.

Table 2 below shows, for each SNP on ECA22 as listed in Table 1, a 60 nucleotide portion of ECA22 containing the SNP. The SNPs are marked in bold and underlining in Table 2 and show the reference allele of the SNPs.

TABLE 2
ECA22 sequence data for the SNPs on ECA22

| SNP Probe Set | Reference | Alternate | | SEQ ID |
|---|---|---|---|---|
| ID | Allele | Allele | ECA22 | NO: |
| AX-102949172 | A | G | (45,532,921-45,532,980): | 1 |
| | | | ctccctccac agcatcagac cgggaaatta | |
| | | | aacccctctg ggctcgtgga gcggagtgag | |
| AX-104791028 | A | G | (45,622,741-45,622,800): | 2 |
| | | | atcaggcacc aaagtccacc aagggcagct | |
| | | | cttggtcgtg tggatgtgag caattagttt | |
| AX-103174786 | T | G | (45,494,401-45,494,460): | 3 |
| | | | atgtgaagga tgctttgtgg ataatgataa | |
| | | | atttagagat gctttccagt tctgtgatgc | |
| AX-103610371 | A | C | (45,471,301-45,471,360): | 4 |
| | | | gctcaatgaa gctcaagccc aggaaatatg | |
| | | | aagagaacta ccacaaaata aaatcaagtg | |
| AX-103572410 | T | G | (45,524,581-45,524,640): | 5 |
| | | | gctgggaggg agggattgag aggactgtgc | |
| | | | tatgaagtat ctgcattaca cacgaagctt | |
| AX-104515875 | G | A | (45,363,001-45,363,060): | 6 |
| | | | caattgtcag gaggagaaga cgttgaacac | |
| | | | gtaactgcaa aaatatttga ccatcaaaag | |
| AX-103805460 | T | C | (45,388,441-45,388,500): | 7 |
| | | | tccacttttg tgcgaaatct cccgctttga | |
| | | | gagaaaagtc aagatgaggg gctctgctac | |
| AX-104362627 | T | C | (45,497,941-45,498,000): | 8 |
| | | | atatatatgc catctaattt tggtttatgc | |
| | | | ttaccacatt tttcaatttt attgaattaa | |
| AX-103795824 | A | G | (45,451,801-45,451,860): | 9 |
| | | | tctgaagcag tggatttcct cttccctggc | |
| | | | tggcttaatc ggaatttccc atccccaagc | |
| AX-103100016 | A | G | (45,500,341-45,500,400): | 10 |
| | | | gaccagatcc caggctggat ctgggcatca | |
| | | | gtggccagct gtggatcacg aaattttcaa | |
| AX-103683359 | T | C | (45,662,701-45,662,760): | 11 |
| | | | aaccacatga aaatagctct ggtcaccaaa | |
| | | | agaacaccaa aaggaacgct aaaacacaag | |
| AX-103453843 | T | C | (45,347,221-45,347,280): | 12 |
| | | | atggctgggg cctccttcac tgcaggacag | |
| | | | agagggggcc accctgagtc tctgccggtg | |
| AX-104157798 | C | T | (45,445,801-45,445,860): | 13 |
| | | | aaactatcta gcaccctcaa caaggtaaaa | |
| | | | tctgtaattc caatttaaaa agcatgaaaa | |
| AX-103739323 | G | A | (45,539,041-45,539,100): | 14 |
| | | | tgtaatgtcc atttcctccc ccacagacac | |
| | | | tgggctgatg acctgcacag tggatgtttg | |
| AX-103902715 | A | G | (45,396,901-45,396,960): | 15 |
| | | | gtgccggttg gtggaagtga ttcattacat | |
| | | | ccagtccaca ccgaagggga ggggaattag | |
| AX-104277754 | A | G | (45,653,581-45,653,640): | 16 |
| | | | tttggatctg tgaggtacct cactacaata | |
| | | | ggtatgagat attcagagaa catcagtaga | |

As is shown in Table 2 the SNP AX-102949172 corresponds to position 11 in SEQ ID NO: 1, SNP AX-104791028 corresponds to position 4 in SEQ ID NO: 2, SNP AX-103174786 corresponds to position 55 in SEQ ID NO: 3, SNP AX-103610371 corresponds to position 57 in SEQ ID NO: 4, SNP AX-103572410 corresponds to position 17 in SEQ ID NO: 5, SNP AX-104515875 corresponds to position 22 in SEQ ID NO: 6, SNP AX-103805460 corresponds to position 55 in SEQ ID NO: 7, SNP AX-104362627 corresponds to position 21 in SEQ ID NO: 8, SNP AX-103795824 corresponds to position 9 in SEQ ID NO: 9, SNP AX-103100016 corresponds to position 8 in SEQ ID NO: 10, SNP AX-103683359 corresponds to position 8 in SEQ ID NO: 11, SNP AX-103453843 corresponds to position 2 in SEQ ID NO: 12, SNP AX-104157798 corresponds to position 14 in SEQ ID NO: 13, SNP AX-103739323 corresponds to position 56 in SEQ ID NO: 14, SNP AX-103902715 corresponds to position 29 in SEQ ID NO: 15 and SNP AX-104277754 corresponds to position 58 in SEQ ID NO: 16.

Thirteen of the SNPs located on ECA22: 45347522-45662708 reached the suggestive threshold (p<1.0×10⁻⁵) corresponding to the thirteen uppermost entries in Table 1. Of these thirteen SNPs, ten were in linkage disequilibrium (LD) (r²≥0.8). These ten SNPs are located in a region of from nucleotide position 45,363,022 to nucleotide position 45,662,708 on ECA22, see Table 3.

Hence, in an embodiment, the at least one biomarker is located in a region of from nucleotide position 45,363,022 to nucleotide position 45,662,708 on ECA22.

TABLE 3
Top SNPs on ECA22 from the GWA analysis for score of back and croup

| | | | | | | | Number | | | |
|---|---|---|---|---|---|---|---|---|---|---|
| SNP Probe | | Position | | | Reference | Alternate | of | | SE of | Raw p- |
| Set ID | ECA | (bp) | A1 | A2 | Allele | Allele | horses | effB | effB | value |
| AX-102949172 | 22 | 45,532,931 | G | A | A | G | 177 | −0.32 | 0.06 | 2.67E−07 |
| AX-104791028 | 22 | 45,622,744 | G | A | A | G | 177 | −0.31 | 0.06 | 6.00E−07 |
| AX-103174786 | 22 | 45,494,455 | G | T | T | G | 177 | −0.30 | 0.06 | 9.66E−07 |
| AX-103610371 | 22 | 45,471,357 | C | A | A | C | 176 | −0.31 | 0.06 | 1.01E−06 |
| AX-103572410 | 22 | 45,524,597 | G | T | T | G | 177 | −0.30 | 0.06 | 1.21E−06 |
| AX-104515875 | 22 | 45,363,022 | G | A | G | A | 177 | 0.29 | 0.06 | 1.83E−06 |
| AX-103805460 | 22 | 45,388,495 | C | T | T | C | 177 | −0.28 | 0.06 | 2.84E−06 |
| AX-103100016 | 22 | 45,500,367 | G | A | A | G | 177 | −0.28 | 0.06 | 8.06E−06 |
| AX-103683359 | 22 | 45,662,708 | C | T | T | C | 177 | −0.28 | 0.06 | 9.06E−06 |
| AX-104157798 | 22 | 45,445,814 | T | C | C | T | 171 | −0.28 | 0.06 | 1.11E−05 |

In an embodiment, determining presence or absence of at least one biomarker comprises determining genotype of at least one SNP selected from the group consisting of the SNPs listed in Table 3.

In a particular embodiment, determining genotype of at least one SNP selected from the group consisting of the SNPs listed in Table 3 comprises determining whether the at least one SNP has the reference allele in Table 3 or the alternate allele in Table 3.

In an embodiment, determining presence or absence of at least one biomarker comprises determining genotype of at least one SNP selected from the group consisting of a SNP located at position 45,363,022 on ECA22, a SNP located at position 45,388,495 on ECA22, a SNP located at position 45,445,814 on ECA22, a SNP located at position 45,471,357 on ECA22, a SNP located at position 45,494,455 on ECA22, a SNP located at position 45,500,367 on ECA22, a SNP located at position 45,524,597 on ECA22, a SNP located at position 45,532,931 on ECA22, a SNP located at position 45,616,738 on ECA22, a SNP located at position 45,622,744 on ECA22, and a SNP located at position 45,662,708 on ECA22.

In a particular embodiment, determining genotype of the at least one SNP comprises determining genotype of a SNP located at position 45,532,931 on ECA22.

In an embodiment, determining genotype of the at least one SNP comprises determining presence or absence of nucleotide A or G at position 45,363,022 on ECA22; determining presence or absence of nucleotide C or T at position 45,388,495 on ECA22; determining presence or absence of nucleotide T or C at position 45,445,814 on ECA22; determining presence or absence of nucleotide C or A at position 45,471,357 on ECA22; determining presence or absence of nucleotide G or T at position 45,494,455 on ECA22; determining presence or absence of nucleotide G or A at position 45,500,367 on ECA22; determining presence or absence of nucleotide G or T at position 45,524,597 on ECA22; determining presence or absence of nucleotide G or A at position 45,532,931 on ECA22; determining presence or absence of nucleotide C or T at position 45,616,738 on ECA22; determining presence or absence of nucleotide G or A at position 45,622,744 on ECA22; and/or determining presence or absence of nucleotide C or T at position 45,662,708 on ECA22.

In a particular embodiment, determining genotype of the at least one SNP comprises determining presence or absence of nucleotide G or A at position 45,532,931 on ECA22.

In another particular embodiment, determining genotype of the at least one SNP comprises determining genotype of at least one SNP selected from the group consisting of a SNP located at position 45,616,738 on ECA22, a SNP located at position 45,622,744 on ECA22 and a SNP located at position 45,662,708 on ECA22.

Determining presence or absence of a particular nucleotide at a given position on ECA22 comprises, in an embodiment, determining the identity of the nucleotide at the given position on ECA22, i.e., determining whether the nucleotide in the given position is adenosine (A), cytosine (C), guanosine (G), or thymidine (T).

The above mentioned embodiments could involve determining presence of a particular nucleotide (A, T, G or C) at a given position on ECA22 in one or both alleles. Alternatively, the above mentioned embodiments could involve determining presence of the particular nucleotide at the given position on ECA22 in one allele. In a further example, the above mentioned embodiments could involve determining presence of the particular nucleotide at the given position on ECA22 in both alleles.

Correspondingly, the above mentioned embodiments could involve determining absence of a particular nucleotide at a given position on ECA22 in one or both alleles. Alternatively, the above mentioned embodiments could involve determining absence of the particular nucleotide at the given position on ECA22 in one allele. In a further example, the above mentioned embodiments could involve determining absence of the particular nucleotide at the given position on ECA22 in both alleles.

In a particular embodiment, determining genotype of the at least one SNP comprises determining presence of nucleotide A at position 45,363,022 on ECA22 in one or both alleles, in one allele or in both alleles.

In another particular embodiment, determining genotype of the at least one SNP comprises determining presence of nucleotide C at position 45,388,495 on ECA22 in one or both alleles, in one allele or in both alleles.

In a further particular embodiment, determining genotype of the at least one SNP comprises determining presence of nucleotide T at position 45,445,814 on ECA22 in one or both alleles, in one allele or in both alleles.

In yet another particular embodiment, determining genotype of the at least one SNP comprises determining presence of nucleotide C at position 45,471,357 on ECA22 in one or both alleles, in one allele or in both alleles.

In a particular embodiment, determining genotype of the at least one SNP comprises determining presence of nucleotide G at position 45,494,455 on ECA22 in one or both alleles, in one allele or in both alleles.

In another particular embodiment, determining genotype of the at least one SNP comprises determining presence of nucleotide G at position 45,500,367 on ECA22 in one or both alleles, in one allele or in both alleles.

In a further particular embodiment, determining genotype of the at least one SNP comprises determining presence of nucleotide G at position 45,524,597 on ECA22 in one or both alleles, in one allele or in both alleles.

In yet another particular embodiment, determining genotype of the at least one SNP comprises determining presence of nucleotide G at position 45,532,931 on ECA22 in one or both alleles, in one allele or in both alleles.

In a particular embodiment, determining genotype of the at least one SNP comprises determining presence of nucleotide G at position 45,622,744 on ECA22 in one or both alleles, in one allele or in both alleles.

In another particular embodiment, determining genotype of the at least one SNP comprises determining presence of nucleotide C at position 45,662,708 on ECA22 in one or both alleles, in one allele or in both alleles.

In further particular embodiments, determining genotype of the at least one SNP comprises determining presence of nucleotide T or C at position 45,616,738 in one or both alleles, in one allele or in both alleles; determining presence of nucleotide G or A at position 45,622,744 in one or both alleles, in one allele or in both alleles; and/or determining presence of nucleotide C or T at position 45,662,708 in one or both alleles, in one allele or in both alleles.

There are several methods known by those skilled in the art for determining whether a particular nucleotide sequence is present in a nucleic acid molecule and for identifying the nucleotide in a given position in a nucleic acid sequence. These include the amplification of a nucleic acid segment encompassing the genetic marker by means of polymerase chain reaction (PCR) or any other amplification method, interrogate the genetic marker by means of allele specific hybridization, 3′-exonuclease assay (Taqman assay), fluorescent dye and quenching agent-based PCR assay, the use of allele-specific restriction enzymes (RFLP-based techniques), direct sequencing, oligonucleotide ligation assay (OLA), pyrosequencing, invader assay, mini-sequencing, denaturing high pressure liquid chromatography (DHPLC) based techniques, single strand conformational polymorphism (SSCP), allele-specific PCR, denaturating gradient gel electrophoresis (DGGE), temperature gradient gel electrophoresis (TGGE), chemical mismatch cleavage (CMC), heteroduplex analysis based system, techniques based on mass spectroscopy (MS), invasive cleavage assay, polymorphism ratio sequencing (PRS), microarrays, rolling circle extension assay, high pressure liquid chromatography (HPLC) based techniques, extension based assays, amplification refractory mutation system (ARMS), Amplification refractory mutation linear extension (ALEX), single base chain extension (SBCE), molecular beacon assays, invader (Third wave technologies), ligase chain reaction assays, 5′-nuclease assay-based techniques, hybridization capillary array electrophoresis (CAE), protein truncation assays (PTT), immunoassays, and solid phase hybridization (dot blot, reverse dot blot, chips). This list of methods is not meant to be exclusive, but just to illustrate the diversity of available methods. Some of these methods can be performed in microarray format (microchips) or on beads.

Haplotype analysis was performed by constructing a LD plot and the ten significant SNPs in LD (r²≥0.8), see Table 2, were used to estimate haplotype. The haplotype effect on the score of back and croup was estimated by a generalized linear model (glm). The most frequent haplotype was used as a reference and only haplotypes with frequencies greater than 0.02 were included. A simulated p-value was estimated by using 100000 permutations considering an additive effect. The haplotype analysis revealed two opposite haplotypes which resulted in higher and lower scores for back and croup (p-value <0.001) (Table 4).

Hence, in an embodiment, determining genotype of the at least one SNP comprises determining whether the horse has a first haplotype or a second haplotype. In this embodiment, the first haplotype comprises nucleotide A at position 45,363,022 on ECA22, nucleotide C at position 45,388,495 on ECA22, nucleotide T at position 45,445,814 on ECA22, nucleotide C at position 45,471,357 on ECA22, nucleotide G at position 45,494,455 on ECA22, nucleotide G at position 45,500,367 on ECA22, nucleotide G at position 45,524,597 on ECA22, nucleotide G at position 45,532,931 on ECA22, nucleotide G at position 45,622,744 on ECA22 and nucleotide C at position 45,662,708 on ECA22. The second haplotype comprises, in this embodiment, nucleotide G at position 45,363,022 on ECA22, nucleotide T at position 45,388,495 on ECA22, nucleotide C at position 45,445,814 on ECA22, nucleotide A at position 45,471,357 on ECA22, nucleotide T at position 45,494,455 on ECA22, nucleotide A at position 45,500,367 on ECA22, nucleotide T at position 45,524,597 on ECA22, nucleotide A at position 45,532,931 on ECA22, nucleotide A at position 45,622,744 on ECA22 and nucleotide T at position 45,662,708 on ECA22.

Haplotype as used herein refers to a set of SNP alleles that are associated statistically.

In a particular embodiment, the first haplotype is associated with a higher score of back and croup and the second haplotype is associated with a lower score of back and croup.

Experimental data revealed that several traits other than conformation of back and croup differed significantly in mean scores between horses with the first haplotype and the second haplotype. The two haplotypes differed significantly in mean scores (p-value 0.05) for the gait traits tölt and pace (Table 5). The two haplotypes also differed significantly in means for the zoometric measurements of depth at breast, width of hips and thigh bones, and length of the forelimbs (Table 5). In addition to this, there were significant differences between the two haplotypes for the sub-traits backline and the croup type (Table 5).

Hence, the first haplotype is associated with a higher gait quality, in particular a higher lateral gait quality and preferably a higher tölt quality and/or pace quality, as compared to the second haplotype.

In an embodiment, determining genotype of the at least one SNP comprises determining whether the horse is homozygote or heterozygote for the first haplotype or homozygote for the second haplotype.

Horses that were homozygote for the first haplotype had higher mean scores for the traits croup and back, tölt, pace, zoometric measurements of depth at breast, width of hips and thigh bones, and length of the forelimbs and for the sub-traits backline and the croup type as compared to horses that were homozygote for the second haplotype (Table 5).

The biomarkers of the present invention also have an association with gait performance type of horses. In more detail, 56 out of 89 pacing horses were homozygous or heterozygous for the haplotype T-G-C of three SNPs, whereas another haplotype C-A-T of these three SNPs was correlated with horses that performed at trot as 80 out of 89 trotting horses were homozygous for this another haplotype. Hence, biomarkers of the invention can be used to predict the performance type of horses and predict whether the horses are trotters, i.e., have high trot quality and performance, or pacers, i.e., have high pace quality and performance.

In this embodiment, the first haplotype T-G-C corresponds to nucleotide T at position 45,616,738 on ECA22, nucleotide G at position 45,622,744 on ECA22 and nucleotide C at position 45,662,708 on ECA22. The second haplotype C-A-T corresponds to nucleotide C at position 45,616,738 on ECA22, nucleotide A at position 45,622,744 on ECA22 and nucleotide T at position 45,662,708 on ECA22.

In a particular embodiment, a horse is predicted to be a trotter, i.e., having high trot quality and performance, if the horse is determined to be homozygous for the second haplotype C-A-T. Correspondingly, a horse is predicted to be a pacer, i.e., having high pace quality and performance, if the horse is determined to be homozygous or heterozygous for the first haplotype T-G-C.

Another aspect of the invention relates to a method for selection a horse for breeding. The method comprises determining, in a sample comprising nucleic acid molecules obtained from the horse, the allele of at least one biomarker useful for predicting conformation of back and croup and/or gait quality and/or gait performance type trot or pace of the horse. The at least one biomarker is located in a region of from nucleotide position 44,000,000 to nucleotide position 47,000,000 on ECA22, preferably from nucleotide position 44,347,522 to nucleotide position 46,662,708 on ECA22, and more preferably from nucleotide position 45,347,522 to nucleotide position 45,662,708 on ECA22, such as from nucleotide position 45,363,022 to nucleotide position 45,662,708 on ECA22. The method also comprises selecting the horse for breeding based on the determined allele of the at least one biomarker.

Hence, the novel QTL of the present invention that is indicative of conformation of back and croup of horses and of gait quality, and in particular lateral gait quality, can be used to select horses for breeding.

The various embodiments described in the foregoing for determining the allele of at least one biomarker or genetic marker, such as determining presence or absence of a particular nucleotide at a given position in ECA22, can also be applied to the present aspect of selecting a horse for breeding.

For instance, the method of the invention could be used to identify and select a stallion that is heterozygote or preferably homozygote for the alternate allele in Table 2 for any SNP in Table 2. Alternatively, or in addition, the method could be used to identify and select a mare that is heterozygote or preferably homozygote for the alternate allele in Table 2 for any SNP in Table 2.

In a particular embodiment, the method comprises determining whether the horse (stallion or mare) is heterozygote, preferably homozygote, for the first haplotype or the second haplotype. In such an embodiment, the method comprises selecting the horse for breeding if the horse is heterozygote, preferably homozygote, for the first haplotype.

In a most preferred embodiment, the method of the invention is used to identify and select a stallion that is homozygote for the first haplotype and identify and select a mare that is homozygote for the first haplotype. The identified and selected stallion and mare can then be mated to get an offspring that will be homozygote for the first haplotype.

In an embodiment, the horse is an Icelandic horse, i.e., a horse of the Icelandic breed.

A further aspect of the invention relates to a method for selecting a training scheme for a horse. The method comprises determining, in a sample comprising nucleic acid molecules obtained from the horse, the allele of at least one biomarker useful in predicting conformation of back and croup and/or gait quality and/or gait performance type trot or pace of the horse. The at least one biomarker is located in a region of from nucleotide position 44,000,000 to nucleotide position 47,000,00 on ECA22, preferably from nucleotide position 44,347,522 to nucleotide position 46,662,708 on ECA22, and more preferably from nucleotide position 45,347,522 to nucleotide position 45,662,708 on ECA22. The method also comprises selecting the training scheme for the horse based on the determined allele of the at least one biomarker.

Hence, in this aspect, the training scheme or schedule of a horse is selected based on the determined allele of the at least one biomarker. Training scheme or schedule as used herein is, in an embodiment, a scheme for training the horse according to a particular gait type. For instance, in a particular embodiment, the training scheme could be adapted to train the horse's trot performance, i.e., a trot training scheme for trotters, or be adapted to train the horse's pace performance, i.e., a pace training scheme for pacers.

In an embodiment, determining the allele of the at least one biomarker comprises determining, in the sample, the allele of at least one biomarker selected from the group consisting of a SNP located at position 45,616,738 on ECA22, a SNP located at position 45,622,744 on ECA22 and a SNP located at position 45,662,708 on ECA22. Selecting the training scheme for the horse comprises, in this embodiment, selecting a training scheme adapted for pacers or a training scheme adapted for trotters based on the determined allele of the at least one biomarker.

In this embodiment, one of the SNPs is determined and used in the selection, such as the SNP located at position 45,616,738 on ECA22, the SNP located at position 45,622,744 on ECA22 or the SNP at position 45,662,708 on ECA22. In another embodiment, two of the SNPs are determined and used in the selection of training scheme, such as the SNPs located at positions 45,616,738 and 45,622,744 on ECA22, the SNPs located at positions 45,616,738 and 45,662,708 on ECA22, or the SNPs located at positions 45,622,744 and 45,662,708 on ECA22. In a further embodiment, all three the SNPs are determined and used in the selection of training scheme.

In a particular embodiment, determining the allele of the at least one biomarker comprises determining, in the sample, whether the horse has a first haplotype or a second haplotype. In this particular embodiment, the first haplotype comprises nucleotide T at position 45,616,738 on ECA22, nucleotide G at position 45,622,744 on ECA22 and nucleotide C at position 45,662,708 on ECA22. The second haplotype comprises nucleotide C at position 45,616,738 on ECA22, nucleotide A at position 45,622,744 on ECA22 and nucleotide T at position 45,662,708 on ECA22. In this particular embodiment, selecting the training scheme for the horse comprises selecting the training scheme adapted for pacers if the horse is homozygous or heterozygous for the first haplotype and selecting the training scheme adapted for trotters if the horse is homozygous for the second haplotype.

A further aspect of the invention relates to a kit for predicting conformation of back and croup and/or gait quality and/or gait performance type trot or pace, in particular lateral gait quality, of a horse. The kit comprises at least one oligonucleotide probe capable of forming a hybridized nucleic acid with a SNP or a nucleic acid region flanking the SNP. The SNP is selected from the group consisting of a SNP located at position 45,363,022 on ECA22, a SNP located at position 45,388,495 on ECA22, a SNP located at position 45,445,814 on ECA22, a SNP located at position 45,471,357 on ECA22, a SNP located at position 45,494,455 on ECA22, a SNP located at position 45,500,367 on ECA22, a SNP located at position 45,524,597 on ECA22, a SNP located at position 45,532,931 on ECA22, a SNP located at position 45,622,744 on ECA22, and a SNP located at position 45,662,708 on ECA22. The kit also comprises instructions for predicting conformation of back and croup and/or gait quality and/or gait performance type trot or pace, in particular lateral gait quality, of the horse based on the horse's genotype at the SNP.

Yet another aspect of the invention relates to a kit for predicting conformation of back and croup and/or gait quality and/or gait performance type trot or pace, in particular lateral gait quality, of a horse. The kit comprises at least one oligonucleotide probe capable of forming a hybridized nucleic acid with a SNP or a nucleic acid region flanking the SNP. The SNP is selected from the group consisting of a SNP located at position 45,616,738 on ECA22, a SNP located at position 45,622,744 on ECA22, and a SNP located at position 45,662,708 on ECA22. The kit also comprises instructions for predicting conformation of back and croup and/or gait quality and/or gait performance type trot or pace, in particular lateral gait quality, of the horse based on the horse's genotype at the SNP.

In an embodiment, the at least one oligonucleotide probe capable of forming a hybridized nucleic acid with a SNP or a nucleic acid region flanking the SNP is at least one first oligonucleotide probe. In this embodiment, the kit further comprises a second oligonucleotide probe capable of forming a hybridized nucleic acid with a SNP defined above or a nucleic acid region flanking a SNP defined above.

In an embodiment, the first oligonucleotide probe is a first primer that hybridizes 5′ or 3′ to a SNP defined above and the second oligonucleotide probe is a second primer that hybridizes 3′ or 5′ to the SNP. In a particular embodiment, the first and second primers are capable of amplifying the SNP, i.e., the first and second primers form a primer pair. Alternatively, one of the first and second primers hybridizes to portion or segment of the nucleic acid sequence comprising the SNP.

The present invention also relates to an isolated nucleic acid comprising a SNP. The SNP is selected from the group consisting of a SNP located at position 45,363,022 on ECA22, a SNP located at position 45,388,495 on ECA22, a SNP located at position 45,445,814 on ECA22, a SNP located at position 45,471,357 on ECA22, a SNP located at position 45,494,455 on ECA22, a SNP located at position 45,500,367 on ECA22, a SNP located at position 45,524,597 on ECA22, a SNP located at position 45,532,931 on ECA22, a SNP located at position 45,616,738 on ECA22, a SNP located at position 45,622,744 on ECA22, and a SNP located at position 45,662,708 on ECA22, preferably selected from the group consisting of a SNP located at position 45,363,022 on ECA22, a SNP located at position 45,388,495 on ECA22, a SNP located at position 45,445,814 on ECA22, a SNP located at position 45,471,357 on ECA22, a SNP located at position 45,494,455 on ECA22, a SNP located at position 45,500,367 on ECA22, a SNP located at position 45,524,597 on ECA22, a SNP located at position 45,532,931 on ECA22, a SNP located at position 45,622,744 on ECA22, and a SNP located at position 45,662,708 on ECA22 or selected from the group consisting of a SNP located at position 45,616,738 on ECA22, a SNP located at position 45,622,744 on ECA22, and a SNP located at position 45,662,708.

### EXAMPLES

### Example 1

This Example provides valuable information about the genetics of conformation of the back and croup in Icelandic horses. A novel QTL for the trait back and croup was detected on ECA22: 45347522-45662708. The QTL is associated with the back inclination, the form of the croup, and length of limbs as well as the quality of the lateral gaits pace and tölt. The QTL contributes to an uphill conformation, where the frontal part of the horse is higher than the hind part, and a strong backline. Together, this gives the horse a more well-balanced backline with an increased carrying ability. The QTL also contributes to straight movements of the horse, in that the horse is more evenly strong in the left and right side. This means that the horse, already from the beginning of training, has a natural talent for being trained under rider. Therefore, the horse also has a higher potential to become a valuable riding horse. Overall, the contributions of this QTL to straightness and balance are especially important for lateral gaits such as tölt and pace in the horse. An uphill conformation and a light frontal part of the horse is especially beneficial for the highly valued gait tölt. These findings could be used as a basis for a genetic test to aid in the selection of breeding horses, thus, they are of major interest for horse breeders.

Results

Genome-Wide Association (GWA) Analysis for Conformation of Back and Croup

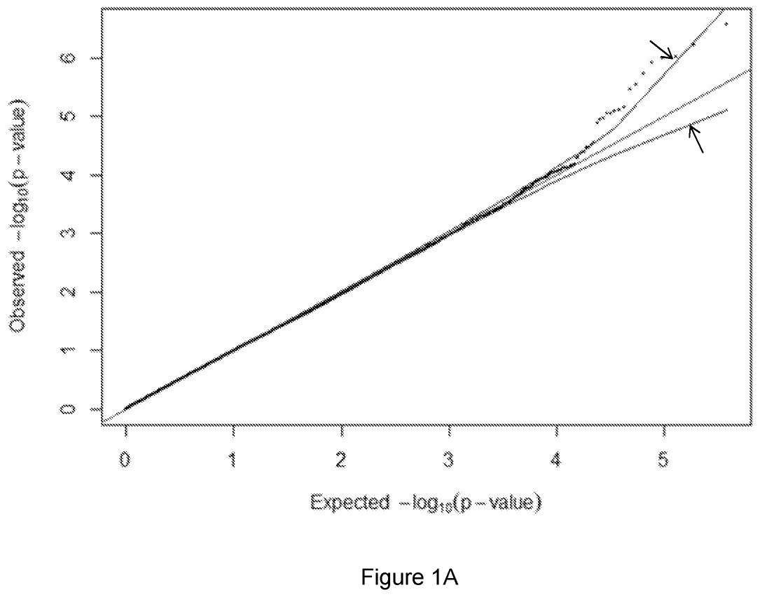

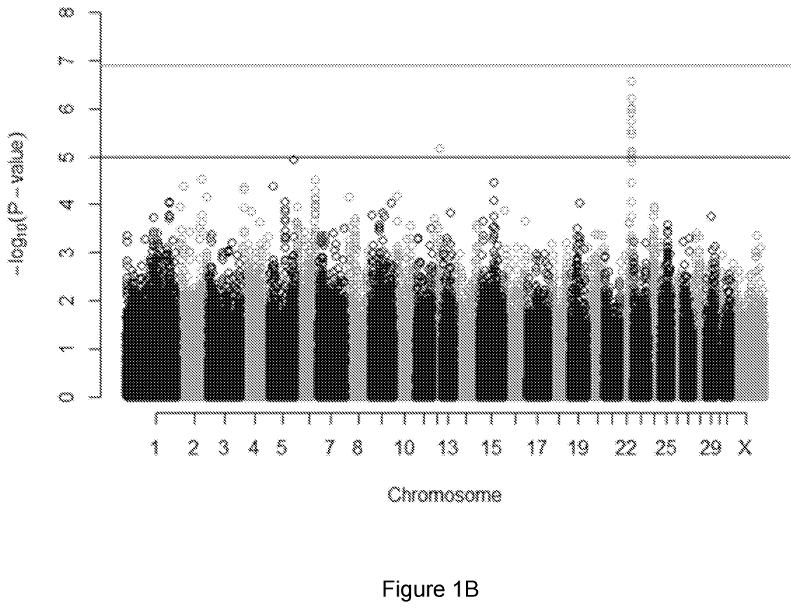

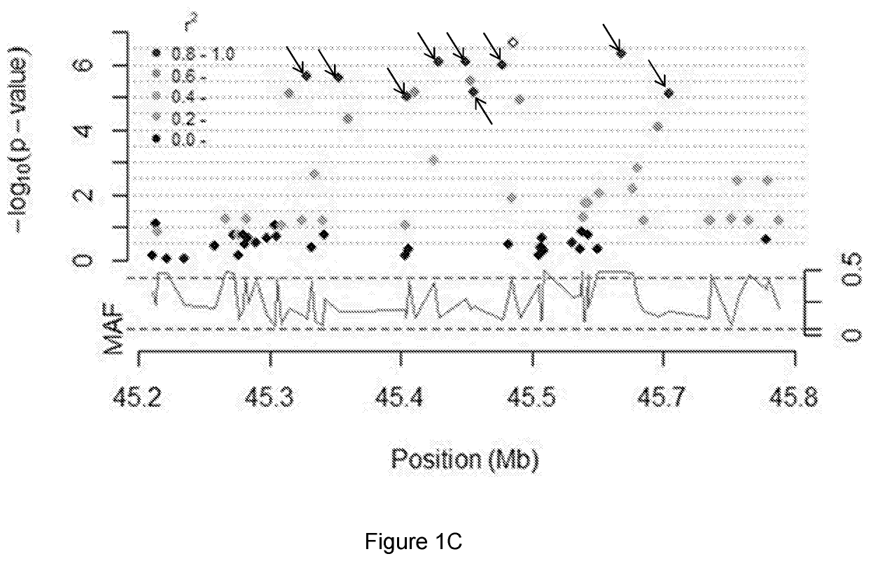

In total, 383896 SNPs (373041 autosomal and 10855 X chromosomal) and 177 horses passed quality control (QC) and were included in the GWA analysis. Thirteen SNPs located on ECA22: 45347522-45662708 reached the suggestive threshold (p<1.0×10⁻⁵), of which ten were in linkage disequilibrium (LD) (r²≥0.8) (FIG. 1). Additionally, one single SNP reached the suggestive threshold on ECA12 (FIG. 1). A summary of the GWA results for the 50 top SNPs is presented in Table 4.

TABLE 4
Summary of the 50 top SNPs from the GWA analysis for score of back and croup

| SNP Probe | | Position | | | Reference | Alternate | Number | | SE of | Raw |
|---|---|---|---|---|---|---|---|---|---|---|
| Set ID | ECA | (bp) | A1 | A2 | Allele | Allele | of horses | effB | effB | p-value |
| AX-102949172 | 22 | 45532931 | G | A | A | G | 177 | −0.32 | 0.06 | 2.67E−07 |
| AX-104791028 | 22 | 45622744 | G | A | A | G | 177 | −0.31 | 0.06 | 6.00E−07 |
| AX-103174786 | 22 | 45494455 | G | T | T | G | 177 | −0.30 | 0.06 | 9.66E−07 |
| AX-103610371 | 22 | 45471357 | C | A | A | C | 176 | −0.31 | 0.06 | 1.01E−06 |
| AX-103572410 | 22 | 45524597 | G | T | T | G | 177 | −0.30 | 0.06 | 1.21E−06 |
| AX-104515875 | 22 | 45363022 | G | A | G | A | 177 | 0.29 | 0.06 | 1.83E−06 |
| AX-103805460 | 22 | 45388495 | C | T | T | C | 177 | −0.28 | 0.06 | 2.84E−06 |
| AX-104362627 | 22 | 45497961 | C | T | T | C | 177 | −0.28 | 0.06 | 3.45E−06 |
| AX-103800701 | 12 | 26756656 | T | C | T | C | 177 | 0.27 | 0.06 | 6.92E−06 |
| AX-103795824 | 22 | 45451809 | G | A | A | G | 176 | −0.28 | 0.06 | 7.65E−06 |
| AX-103100016 | 22 | 45500367 | G | A | A | G | 177 | −0.28 | 0.06 | 8.06E−06 |
| AX-103683359 | 22 | 45662708 | C | T | T | C | 177 | −0.28 | 0.06 | 9.06E−06 |
| AX-103453843 | 22 | 45347522 | T | C | T | C | 177 | −0.27 | 0.06 | 9.09E−06 |
| AX-104157798 | 22 | 45445814 | T | C | C | T | 171 | −0.28 | 0.06 | 1.11E−05 |
| AX-104378639 | 5 | 92718241 | A | G | G | A | 176 | 0.27 | 0.06 | 1.16E−05 |
| AX-103739323 | 22 | 45539096 | A | G | G | A | 177 | −0.27 | 0.06 | 1.30E−05 |
| AX-104868392 | 2 | 96122175 | G | A | G | A | 168 | 0.35 | 0.08 | 2.87E−05 |
| AX-103808861 | 6 | 75218461 | A | G | A | G | 177 | −0.28 | 0.07 | 3.12E−05 |
| AX-104853604 | 15 | 55551672 | C | A | C | A | 177 | −0.25 | 0.06 | 3.43E−05 |
| AX-103902715 | 22 | 45396929 | G | A | A | G | 176 | −0.25 | 0.06 | 3.44E−05 |
| AX-102999535 | 5 | 12582669 | G | A | G | A | 176 | 0.57 | 0.14 | 4.15E−05 |
| AX-104343278 | 2 | 27450484 | T | C | T | C | 176 | −0.32 | 0.08 | 4.18E−05 |
| AX-104069322 | 4 | 13340724 | A | C | A | C | 177 | −0.36 | 0.09 | 4.24E−05 |
| AX-103965917 | 4 | 13355657 | A | G | A | G | 176 | −0.36 | 0.09 | 4.87E−05 |
| AX-104092276 | 6 | 74923675 | G | A | G | A | 177 | −0.27 | 0.07 | 5.12E−05 |
| AX-104545325 | 10 | 12870503 | G | T | T | G | 177 | −0.25 | 0.06 | 6.56E−05 |
| AX-103491474 | 8 | 17851953 | T | C | T | C | 176 | 0.26 | 0.07 | 6.80E−05 |
| AX-103899921 | 2 | 117114323 | T | C | T | C | 176 | −0.45 | 0.11 | 7.01E−05 |
| AX-103018401 | 6 | 74979899 | A | G | A | G | 177 | −0.26 | 0.07 | 7.44E−05 |
| AX-103564499 | 6 | 74974118 | G | A | G | A | 177 | −0.26 | 0.07 | 7.44E−05 |
| AX-103695006 | 6 | 75057845 | T | C | T | C | 177 | −0.27 | 0.07 | 7.52E−05 |
| AX-103100377 | 6 | 74902003 | T | C | T | C | 176 | −0.28 | 0.07 | 7.58E−05 |
| AX-103071980 | 6 | 74859023 | A | C | A | C | 177 | −0.28 | 0.07 | 7.78E−05 |
| AX-104316522 | 6 | 74874325 | G | A | G | A | 177 | −0.28 | 0.07 | 7.78E−05 |
| AX-103666786 | 15 | 55559137 | A | G | G | A | 176 | 0.26 | 0.07 | 8.43E−05 |
| AX-104277754 | 22 | 45653638 | G | A | A | G | 176 | −0.24 | 0.06 | 8.54E−05 |
| AX-104697836 | 1 | 160046196 | G | A | A | G | 177 | 0.28 | 0.07 | 8.76E−05 |
| AX-103333793 | 5 | 59821567 | G | A | A | G | 177 | −0.25 | 0.06 | 8.77E−05 |
| AX-103232247 | 1 | 160052271 | G | A | A | G | 177 | 0.28 | 0.07 | 8.79E−05 |
| AX-104239608 | 19 | 33467486 | C | T | C | T | 177 | −0.40 | 0.10 | 9.00E−05 |
| AX-103255629 | 1 | 160044693 | A | G | G | A | 175 | 0.28 | 0.07 | 9.09E−05 |
| AX-104193488 | 1 | 160048209 | G | T | T | G | 176 | 0.28 | 0.07 | 9.18E−05 |
| AX-103812654 | 9 | 75661302 | A | G | A | G | 175 | 0.25 | 0.06 | 9.35E−05 |
| AX-103588703 | 6 | 75301348 | A | C | A | C | 174 | −0.25 | 0.06 | 9.53E−05 |
| AX-103392867 | 6 | 74966733 | G | T | G | T | 176 | 0.26 | 0.07 | 1.03E−04 |
| AX-103183772 | 6 | 6683969 | G | A | G | A | 177 | 0.25 | 0.06 | 1.08E−04 |
| AX-103421036 | 24 | 23207512 | G | A | G | A | 177 | −0.36 | 0.09 | 1.10E−04 |
| AX-103717705 | 24 | 23178038 | A | G | A | G | 177 | −0.36 | 0.09 | 1.10E−04 |
| AX-104554642 | 24 | 23200837 | G | A | G | A | 177 | −0.36 | 0.09 | 1.10E−04 |
| AX-104068747 | 2 | 16865279 | G | A | A | G | 177 | −0.30 | 0.08 | 1.12E−04 |

SNP Probe Set ID of SNP from Axiom ™ Equine Genotyping Array annotation file
ECA Equus caballus chromosome
A1 major allele
A2 minor allele
Reference Allele refers to the base that is present in the horse reference genome
Alternate Allele refers to any base other than the allele present in the reference genome
effB estimated allele substitution effect
SE of effB standard error of the effB

Haplotype Analysis

The haplotype analysis revealed two opposite haplotypes, which resulted in higher and lower scores for back and croup (p-value <0.001) (Table 5). 34 horses were homozygous for the haplotype associated with a higher score and 28 horses homozygous for the haplotype associated with a lower score of back and croup.

TABLE 5
Results from haplotype analysis for the score of back and croup.

| Haplotypes (SNPs numbers^{*}) | | | | | | | | | | | | | Sim. |
|---|---|---|---|---|---|---|---|---|---|---|---|---|---|
| 1 | 2 | 3 | 4 | 5 | 6 | 7 | 8^{*} | 9 | 10 | Coef | Freq | p-value | p-value |
| G | T | C | A | T | A | T | A | A | T | −0.300 | 0.383 | <0.001 | <0.001 |
| G | T | C | A | T | A | T | A | G | C | 0.090 | 0.021 | 0.657 | 0.718 |
| G | T | C | A | G | G | G | A | A | T | 0.119 | 0.027 | 0.518 | 0.889 |
| G | C | T | C | T | A | G | A | A | T | 0.090 | 0.025 | 0.626 | 0.963 |
| A | C | T | C | G | G | G | G | G | C | 0.300 | 0.474 | <0.001 | <0.001 |

*SNP numbers in bp position order with top SNP as number 8 with reference allele A and alternate allele G (SNP1: AX-104515875; SNP2: AX-103805460; SNP3: AX-104157798; SNP4: AX-103610371; SNP5: AX-103174786; SNP6: AX-103100016; SNP7: AX-103572410; SNP8: AX-102949172; SNP9: AX-104791028; SNP10: AX-103683359)
Coef. = coefficient, estimated effect of the haplotype on the score of back and croup from the generalized linear model (glm) in the haplotype analysis
Freq. = frequencies
Sim. p-value = p-value adjusted by using 100000 permutations
Significant results in bold

Phenotype Association of the Haplotypes with a Significant Effect on the Score of Back and Croup

The t-test analyses revealed that several traits in addition to back and croup significantly differed in mean scores between horses with the favorable and unfavorable haplotype. The two haplotype groups differed significantly in mean scores (p-value 0.05) for the gait traits tölt and pace (Table 6). The two haplotype groups also differed significantly in means for the zoometric measurements of depth at breast, width of hips and thigh bones, and length of the forelimbs. In addition to this, there were significant differences between the two haplotype groups for the sub-traits backline and the croup type.

TABLE 6
Significant results from t-test comparing phenotypes
in horses with different haplotypes.

| | Favorable | | Unfavorable | | | | |
|---|---|---|---|---|---|---|---|
| | haplotype | | haplotype | | | | |
| Trait | N | Mean | N | Mean | t-value | df | p-value |
| Back and croup | 34 | 8.29 | 28 | 7.71 | 4.05 | 58.08 | <0.001 |
| Tölt¹ | 33 | 8.41 | 27 | 7.96 | 2.52 | 45.79 | 0.015 |
| Pace¹ | 33 | 7.18 | 27 | 6.09 | 2.99 | 50.24 | 0.004 |
| Slow tölt¹ | 33 | 8.14 | 26 | 7.73 | 2.14 | 45.19 | 0.038 |
| Depth at breast (M4)² | 33 | 63.2 | 28 | 64.6 | −3.52 | 56.22 | 0.001 |
| Width of the hips (M7)² | 23 | 47.0 | 20 | 48.1 | −2.21 | 37.54 | 0.033 |
| Width between thigh bones (M8)² | 23 | 43.0 | 20 | 44.2 | −2.23 | 38.86 | 0.031 |
| Length of forelimbs (M1-2 × M4)² | 33 | 15.2 | 28 | 12.1 | 3.22 | 40.81 | 0.003 |
| Backline³ | 34 | 1.79 | 28 | 2.25 | −2.69 | 58.91 | 0.009 |
| Croup type³ | 34 | 1.85 | 28 | 2.18 | −2.31 | 53.23 | 0.025 |

¹Subjectively assessed traits (scale 5-10)
²Zoometric measurements (cm)
³Subjectively assessed sub-traits (scale 1-3)
N = Number of horses

Allele Frequency of Top SNP and Doublesex and Mab-3 Related Transcription Factor (DMRT3) in Different Breeds

Comparing allele frequencies of the top SNP identified from GWA analysis (AX-102949172) between different breeds revealed a higher frequency of the alternate allele (the favorable allele) in the Icelandic breed compared with all other investigated breeds (Table 7).

TABLE 7
Allele frequency of top SNP for back and croup and DMRT3

| | Top SNP | | | DMRT3 | | |
|---|---|---|---|---|---|---|
| Breed | N | AF alt | Source | N | AF alt | Source |
| Icelandic horses included | 177 | 0.50 | Array genotyping | 177 | 0.94 | Array genotyping |
| in present Example¹ | | | | | | |
| Icelandic horses unassessed² | 49 | 0.51 | SNP genotyping | 49 | 0.90 | SNP genotyping |
| Other gaited breeds | | | | | | |
| Rocky-Mountain | 36 | 0.33 | SNP genotyping | 27 | 1 | SNP genotyping |
| Colombian trocha | 37 | 0.24 | Array genotyping | 37 | 0.0 | SNP genotyping |
| Colombian trot and gallop | 11 | 0.23 | Array genotyping | 11 | 0.0 | SNP genotyping |
| Colombian paso fino | 38 | 0.29 | SNP genotyping | 28 | 1 | (2) |
| Partly gaited breeds | | | | | | |
| American Curly | 27 | 0.32 | SNP genotyping | 101 | 0.70 | (3) |
| American Saddlebred | 42 | 0.29 | SNP genotyping | 89 | 0.28 | (4) |
| Morgan | 30 | 0.44 | SNP genotyping | 59 | 0.14 | (3) |
| Non-gaited breeds | | | | | | |
| Exmoor | 279 | 0.01 | (5) | 27 | 0.0 | (5) |
| Connemara Pony | 40 | 0.05 | (6) | 35 | 0.0 | (5) |
| Swedish Warmblood | 379 | 0.26 | (7) | 64 | 0.0 | (4, 8) |
| Thoroughbred racehorses | 370 | 0.14 | (9) | 55 | 0.0 | (4, 8) |
| Persian-Arabian horses | 101 | 0.32 | (10) | 69 | 0.0 | (4) |
| North-Swedish draught | 25 | 0.38 | (11) | 34 | 0.0 | (4, 8) |
| Harness racing breeds | | | | | | |
| Coldblooded trotters | 565 | 0.13 | (12) | 306 | 0.45 | (13) |
| Standardbred | 40 | 0.29 | SNP genotyping | 270 | 0.97 | (4, 8) |

N = number of horses included in dataset
Top SNP = the top SNP identified from the GWA analysis for back and croup (AX-102949172)
AF alt = frequency of alternate allele
DMRT3 AF alt = allele frequency of the alternate allele A in the DMRT3 gene known as the ″Gait Keeper″ mutation
¹The 177 Icelandic horses included in the present Example
²Icelandic horses used for riding but that had not attended breeding field test

Functional Annotation of Genes in the Region Associated with the Score of Back and Croup

The detected QTL ECA22: 45347522-45662708 harbors the genes Chromosome 22 C20orf85 homolog (C22H20orf85), Ankyrin repeat domain 60 (ANKRD60) and LOC100056167 described as serine/threonine-protein phosphatase 4 regulatory subunit 1. The SNP on ECA12 (position 26756656-26756656) was located close to the gene solute carrier family 22 member 8 (SLC22A8). None of the significant SNPs (on ECA12 and 22) overlapped any known QTL for conformation in horses (14).

Discussion

Conformation of the back and croup plays an important role for riding ability, gait ability, welfare, and longevity of the horse (15-18). The present Example was performed to identify genomic regions associated with conformation of the back and croup in Icelandic horses and investigate their effects on riding ability. A novel QTL was detected on ECA22 with candidate genes associated with scoliosis and anthropometric traits in humans. The results show that this QTL is of importance not only for conformation of back and croup, but also for riding ability traits, especially lateral gait quality, in Icelandic horses.

Possible Links Between Scoliosis, Motor Laterality and Lateral Gaits

The detected QTL for the trait back and croup harbors the genes C22H20orf85 and ANKRD60, both of which are potentially linked to adolescent idiopathic scoliosis (AIS) in humans (19). Scoliosis is defined as a lateral curvature of the spine and it is the most common vertebral disorder in children and adolescents. In humans, scoliosis can be caused by muscular dystrophy or cerebral palsy, but the cause is usually unknown and therefore referred to as idiopathic. AIS in humans has been shown to result in a generalized skeletal muscle weakness, respiratory impairment and exercise limitation. Studies on scoliosis in humans have also shown correlation between handedness and truncal asymmetry and that molecular basis of handedness are more likely formed by spinal gene expression asymmetries rather than in the motor cortex. Symptoms of scoliosis in horses has been described as an S-shaped bend of the caudal thoracic vertebral column, resulting in restricted movements of the hind limbs and inflexibility of the back (20). Another report described symptoms as a lateral deviation of the head and cervical and cranial thoracic vertebral column to one side, and associated rotation of the thoracic vertebrae. These deviations result in difficulties for a horse to walk in a straight line (21). However, severe thoracic vertebral malformations in horses are infrequent, and mild to moderate forms of scoliosis may go undetected as the strong dorsal spinal musculature can mask subtle deviations of the vertebral column (21). Scores for conformation of back and croup in horses involve both muscular and skeletal assessments, which may indicate that the back and croup phenotype shares some features with mild forms of scoliosis. It is well known that horses commonly demonstrate motor laterality (handedness) (22-24) and some even have difficulties walking in a straight line at the beginning of training. The latter often need more time in training to improve their balance and straightness.

In general, disorders of the back appear to be relatively common in horses and lead to pain and decreased performance (20). However, to our knowledge, there are no studies reporting the prevalence of back problems or scoliosis in Icelandic horses, and it is generally hard to diagnose back pain in horses. The effect of the QTL is more likely related to functional advantage or disadvantage for movements and strength of the back and croup in horses rather than the result of more severe dysfunctions and pain. This is supported by the relatively high frequency of the unfavorable haplotype among the Icelandic horses in the present Example.

Top SNP Allele Frequency in Other Breeds

Icelandic horses had a higher frequency of the alternate allele (the favorable allele) of the top SNP for back and croup compared with all other investigated breeds, including the other gaited and partly gaited breeds. In addition, the Icelandic horses with the favorable haplotype had on average higher scores for the lateral gaits tölt and pace. Therefore, it is likely that the quality of the lateral gaits rather than the ability to perform the gaits is affected by the QTL. Almost all Icelandic horses carry at least one copy of the mutant allele A in the DMRT3 gene known as the “Gait Keeper” mutation (4, 8). This mutation is known to affect the pattern of locomotion in horses and the ability to perform lateral gaits (8). The Icelandic horses in the present Example had a high frequency of the DMRT3 “Gait Keeper” mutation (0.94), 157 of the 177 horses were homozygous AA. The DMRT3 genotype was taken into account in the phenotype association analysis. Pace scores in horses with the CA genotype were considered as a missing value. Despite this, the Icelandic horses with the favorable haplotype had higher scores for pace. This further supports the hypothesis that the detected QTL affects the quality and not the ability of lateral gaits. The genotyped gaited breed Rocky-Mountain Horse is known to be fixed for the DMRT3 “Gait Keeper” mutation (4). The other genotyped gaited breeds American Curly, American Saddlebred and Morgan horses have a moderate high frequency of the DMRT3 “Gait Keeper” mutation (4, 8, 25). These breeds are considered as partly gaited as not all horses within the breed perform ambling gaits. Trotters are also known to perform lateral gaits, and the reported frequency of the DMRT3 mutation is high in Standardbreds (0.97-1.00) (4, 8) and relatively high in Coldblooded trotters (0.45) (4). All of these gaited and partly gaited breeds had a higher frequency of the reference allele than the alternate allele for the top SNP of back and croup. The genotyped Colombian paso horses (CPH) included a group of horses that perform trocha and one group that only perform trot and gallop. The trocha gait is defined as a four-beat gait that includes a lateral step but it is diagonally coupled and therefore not considered a lateral gait (2, 26). The allele frequency of the top SNP did not differ between these two groups. A group of CPH that perform the lateral gait paso fino was also genotyped. However, like all the other genotyped breeds, this group had a lower frequency of the alternate allele of the top SNP for back and croup compared to the Icelandic horses. None of the other genotyped breeds in this Example segregates for the DMRT3 mutation (4, 8), nor do they perform lateral gaits.

The 49 unassessed Icelandic horses had a similar allele frequency of the top SNP for back and croup as well as for the DMRT3 mutation as the 177 assessed Icelandic horses included in the present Example. The unassessed group included riding school horses and horses used for hobby riding. It could be argued that balance and straightness is even more essential for the training of Icelandic horses as they carry relatively heavy (adult) riders, relative to their size, in lateral gaits such as tölt and pace with strong focus on the gait quality. In addition, the Icelandic horses with the favorable haplotype had higher average scores for the lateral gaits tölt and pace, which are highly valued traits in the breed. It is likely that there has been selection for the alternate allele of the top SNP in Icelandic horses.

Genes within the QTL Associated with Musculoskeletal Traits

The gene ANKRD60 is associated with body height in humans (27) and a recent study in American Miniature Horses reported a QTL for withers height close to another Ankyrin Repeat Domain gene ANKRD1 (28). The QTL region on ECA22 harbors the gene LOC100056167 that is not well annotated in horses. The gene is described as serine/threonine-protein phosphatase 4 regulatory subunit 1 and appears to blast with the pseudogene protein phosphatase 4 regulatory subunit 1 like (PPP4R1L) in humans with 84.17% identity (29). The pseudogene PPP4R1L is transcribed in humans and LOC100056167 has exons. PPP4R1L has a potential effect on bone mineral density as it has a protein phosphatase regulator activity (30). PPP4R1L is regulated by an enhancer (Genehancer ID GH20J058887) with potential implications on body height and BMI-adjusted waist circumference in humans (31,32). Therefore, it is possible that the detected QTL affects both the muscular and skeletal system.

The horses with the favorable haplotype in the present Example had longer forelimbs than those with the unfavorable haplotype. This may be explained, at least to some extent, by the effects of the genes ANKRD60 and LOC100056167. According to a previous Example, high-class Icelandic horses are distinguished from low-class horses by an uphill conformation (16). High-class horses have higher withers and higher set neck and back, compared to height at croup and tuber coxae (16). Uphill conformation is believed to facilitate ease of collection and lightness in the front part, features that are taken into account when gait quality is subjectively assessed at breeding field tests (33). Stride length is associated with limb length in horses and other species (34-36) and stride length is also taken into account when assessing the gait quality at breeding field tests (33). Consequently, stride length and uphill conformation are important factors for higher gait quality scores, both of which may be connected to longer forelimbs. This further supports the results from this Example as the horses with the favorable haplotype had both longer forelimbs and higher scores for tölt and pace. In line with this, the horses with the unfavorable haplotype also had a deeper breast and more negative standardized marks for the sub-trait backline compared with the ones with the favorable haplotype. This indicates that a downhill conformation is more common in horses with the unfavorable haplotype. It is possible that a downhill inclination creates an imbalance between the front and back of the horse, causing difficulties for the horse to stretch the hind legs forward, thus losing the ability for self-carriage and collection. This may also result in a shorter stride length, causing lower scores for tölt and pace.

Length and form of the croup are also known to discriminate between high-class and low-class Icelandic horses (16). In the present Example, horses with the favorable haplotype had more positive standardized marks for the sub-trait croup type. This trait is defined as how evenly the croup is shaped and suggests that the haplotype does not influence the length or inclination of the croup, but only the shape of it. The difference between the two haplotype groups for the width of hips (M7) and width between the thighbones (M8) suggest that horses with the favorable haplotype may have a slimmer framed croup than horses with the unfavorable haplotype.

Complexity of the Phenotype

Until around year 2010, a soft, lower backline was considered to be favorable for the assessment of back and croup of Icelandic horses, as a low position of the back was assumed desirable for tölt (33). A study in American Saddlebred horses detected a region on ECA20 associated with extreme lordosis (swayback) (37). However, in the present Example no significant association with back and croup was detected on ECA20. Horses with the haplotype associated with lower score of back and croup were more inclined to have a forward sloping and/or swayback backline.

The back and croup is a complex trait, with muscular as well as skeletal features of both the back and the croup subjectively assessed and scored together as a single trait. The results show that the novel detected QTL associated with back and croup conformation influences various riding ability and conformation traits.

Methods

Animals

In total, 177 Icelandic horses (77 males and 100 females) born between 1993 and 2014 were included in the Example. Hair samples were collected at breeding field tests and by visiting trainers and breeders in Iceland and Sweden. A few samples were also sent in by horse owners after personal contact and posting on social media. The horses were not specifically selected based on conformation of back and croup.

Pedigree data were obtained from the international Icelandic horse database Worldfengur (38). Maximum relatedness between horses was limited to half-siblings.

Phenotyping

Phenotype data were obtained from the international Icelandic horse database Worldfengur (38). The phenotype used for the genome-wide association (GWA) analysis consisted of the subjectively assessed score for back and croup recorded at breeding field tests between 2000 and 2018. Additional conformation and riding ability traits assessed at breeding field tests were used to investigate the effects of genomic regions detected from GWA analysis. 115 of the 177 horses had attended more than one breeding field test. For these horses, information from the latest assessment was used. The majority of horses were assessed in year 2018 (n=89). The horses were assessed in Iceland (n=81), Sweden (n=87), Germany (n=3), Denmark (n=2) and Norway (n=4). Icelandic horses can attend breeding field test from when they are four years old. The age of assessment was on average 6.7 years and ranged from 4 to 15 years. In our sample, 173 horses were assessed for both conformation and riding ability traits, and 4 horses were only assessed for conformation traits as the ridden test is optional. Pace scores for horses with the CA genotype for the DMRT3 gene (n=20) were treated as missing values.

Back and Croup

Back and croup, along with other conformation and riding ability traits assessed at breeding field tests, were subjectively scored on a scale from 5 to 10 with 0.5 intervals, where a score of 5 was only given if a trait was not presented. Assessment of the trait back and croup comprises several aspects of the conformation of the back, croup and loins. The slope and shape of the backline, which is defined as the line from the base of withers to the lumbosacral joint, were assessed. Length and slope of the croup were also assessed, as well as the width and muscularity of the back, the length and width of the loins and the form and muscularity of the croup (33). A high score for back and croup represents a strong, well-balanced backline and a well-muscled wide back. The croup should be long, evenly formed, well-muscled and adequately sloping. A low score is associated with a swayback, stiff or forward sloping backline, a too short or too long and/or unevenly formed croup and poorly muscled back and croup (33). When the judging panel has reached a consensus on a score for back and croup according to the judging scale, they have the possibility to use standardized marks to describe the most prominent positive and/or negative attributes of the trait.

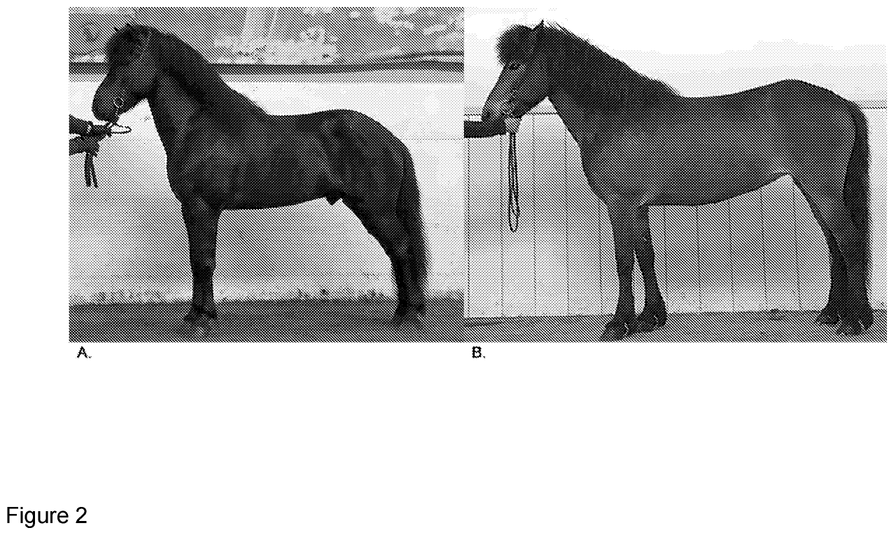

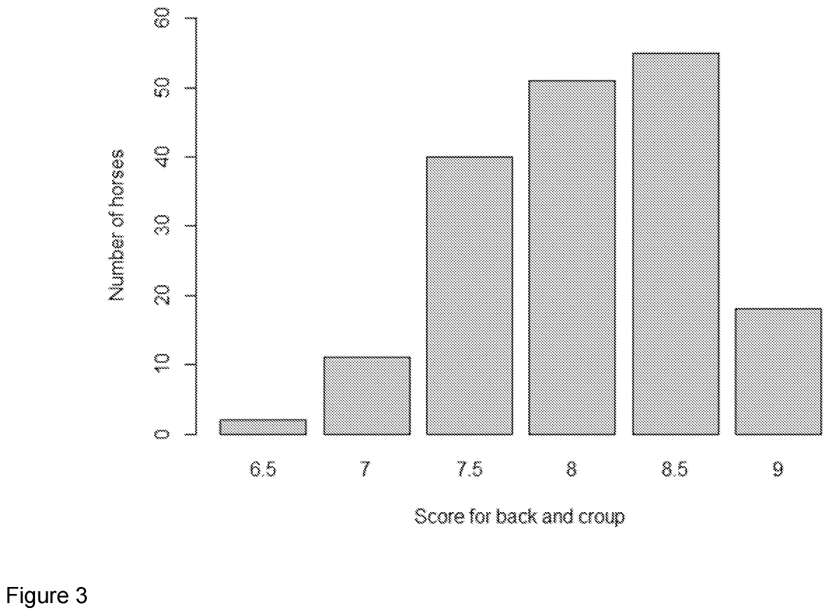

Pictures with examples of horses representing high and low score for back and croup are presented in FIG. 2. The 177 horses in the Example had a score of back and croup that ranged from 6.5 to 9.0 with a mean value of 8.1 (standard deviation (SD) 0.56) (FIG. 3). The distribution of the scores for back and croup was slightly negatively skewed (coefficient of skewness −0.36). Transformation of the raw data to increase normality was tested but was found to not affect the results. Moreover, the residuals from the linear models were normally distributed (results not presented).

Sub-Traits Based on Standardized Marks for Back and Croup

For the purpose of more detailed analysis of the score for back and croup, the standardized marks used to describe prominent positive and negative attributes of the trait were defined as two different sub-traits; backline and croup type. These sub-traits were analysed on a linear scale ranging from 1-3, where 1 represented a positive mark, 3 represented a negative mark and 2 represented no mark and was interpreted as an intermediate description of the trait (not positive or negative). A positive mark for the sub-trait backline was given for good backline (well-balanced backline) and the options for negative marks were forward sloping back, straight back, sway back and/or stiff loins. For the sub-trait croup type, a positive mark was given for evenly formed croup and the options for negative marks were rounded croup, narrowing croup, roof-shaped croup and/or coarse croup.

Additional Trait Assessment Scores from Breeding Field Tests

Besides the conformation trait back and croup, scores for the gait traits tölt, slow tölt, trot, pace, gallop, canter and walk and the trait form under rider were included in this Example. Features of each gait, such as beat, suppleness, stride length, leg-action, speed capacity, collection and lightness, were taken into account when assessing the gaits (33). Scores of all these traits were included to investigate the effects of the detected regions from GWA analysis on the trait back and croup.

Zoometric Traits Measured at Breeding Field Tests

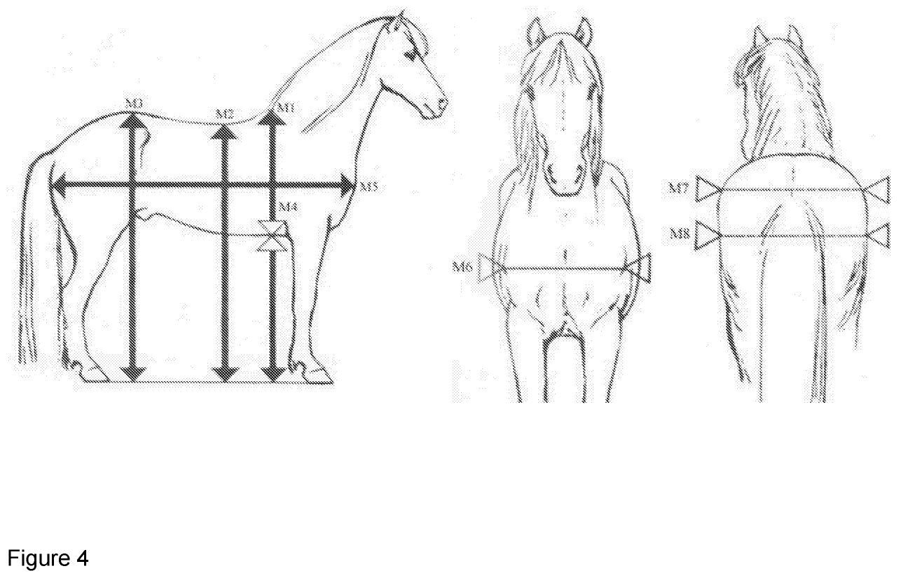

Zoometric measurements are traditionally recorded at breeding field tests to corroborate the subjective conformation assessments (33). All these measurements were included to investigate the effects of the detected genomic regions from GWA analysis for the trait back and croup. The measurements consisted of height at withers (M1), height at lowest point of back (M2), height at croup (M3), depth of breast (M4), length of body (M5), width of chest (M6), width of the hips (M7) and width between thigh bones (M8) (FIG. 4). Length of forelimbs is traditionally assessed from calculation of the difference between height at withers and depth at breast times two (M1-2×M4), as it gives better comparison of the leg length to consider the variation in breast depth between different horses. Other calculated measurements used for conformation assessments were difference between height at withers and height at back (M1-M2), difference between height at withers and height at croup (M1-M3), difference between height at croup and height at back (M3-M2), difference between length of the horse and height at withers (M5-M1), difference between length of the horse and height at croup (M5-M3) and difference between width of hips and width between thigh bones (M7-M8).

DNA Isolation

DNA was extracted from hair roots using a standard procedure of hair preparation. 186 μL of 5% Chelex® 100 Resin (Bio-Rad Laboratories, Hercules, CA) and 14 μL of proteinase K (20 mg/mL; Merck KgaA, Darmstadt, Germany) were added to each sample. This mix was incubated at 56° C. for 2 h at 600 rpm and proteinase K was inactivated for 10 min at 95° C.

Genotyping and Quality Control

The 177 Icelandic horses were genotyped on the 670K+Axiom Equine Genotyping Array. Quality control (QC) was performed with the package GenABEL (39) in R (40) to remove poorly genotyped and noisy data based on the following thresholds: missing genotypes per single nucleotide polymorphism (SNP) (>0.10), missing SNPs per sample (>0.10), minor allele frequency (MAF) (<0.05) and Hardy-Weinberg equilibrium (p-value 1e⁻¹⁰).

Genome-Wide Association Study (GWAS)

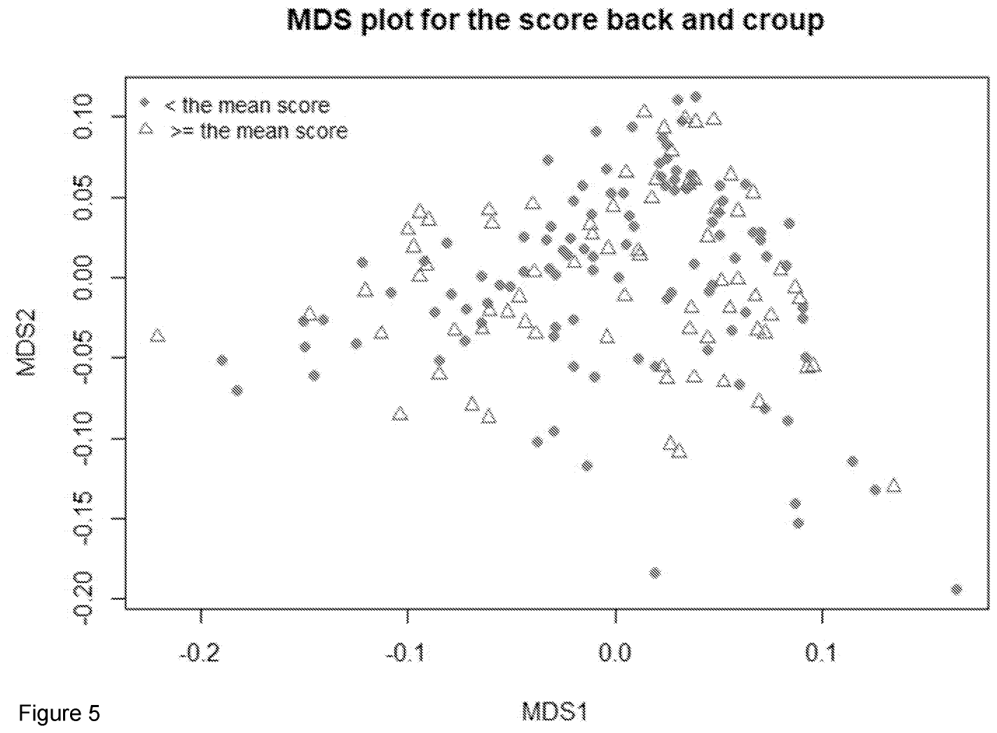

GWA analyses were performed using the package GenABEL (39) in R (71). Possible fixed effects were tested in a linear model using anova as a post hoc test. The tested fixed effects were sex (male or female), age at assessment in age classes (4, 5, 6 or ≥7 years old), age at assessment in years as a linear regression, country of assessment in two classes (Iceland or Sweden/other countries) and year of assessment in five classes (<2010, 2010-2015, 2016, 2017 or 2018). The DMRT3 genotype was also tested as an effect. None of these fixed effects were found to be significant (p 0.05) for the trait back and croup and were thus not included in the GWA models. To investigate potential stratification, a multidimensional scaling (MDS) plot was constructed based on a genomic relationship matrix using the GenABEL package and ibs( ) function (39). No outliers were apparent on the MDS plot and no stratification of horses with low and high score of back and croup was detected. A visualization of the genomic-kinship matrix using MDS is shown in FIG. 5.

The genomic-kinship matrix together with the phenotype of back and croup were passed to the polygenic_hglm function using family gaussian in GenABEL (39,41). To account for any population stratification, the GWA analysis was performed using a mixed model-structured association approach with the mmscore function in GenABEL (39). Genome-wide significance was determined by Bonferroni correction and a suggestive genome-wide significance threshold was set at 1.0×10⁻⁵ (42,43). Quantile-quantile (QQ) and linkage disequilibrium (LD) Manhattan plots were performed using the package cgmisc 2.0 (44).

Haplotype Analysis

Haplotype analysis was performed with the haplo.stats package in R (40). A linkage disequilibrium plot was constructed and the ten significant SNPs in LD (r²≥0.8) were used in the function haplo.em to estimate haplotypes. The haplotype effect on the score of back and croup was estimated by a generalized linear model (glm) with the function haplo.glm. The most frequent haplotype was used as a reference and only haplotypes with frequencies greater than 0.02 were included. A simulated p-value was estimated by using 100000 permutations considering an additive effect.

Phenotype Association of Significant Haplotypes

Phenotype association of the horses homozygous for the haplotypes that had a significant effect on the conformation of back and croup was performed using a two-tailed Student's t-test in R (40). Significance level was set at p-value 0.05. Traits tested were all the zoometric traits, the subjectively scored riding ability traits and the subjectively assessed sub-traits.

Genotyping of the Top SNP and DMRT3 in Other Gaited and Partly Gaited Breeds

Horses of other gaited breeds (Rocky-Mountain: 36 horses, Colombian paso fino horses: 38 horses) and partly gaited breeds (American Curly: 27 horses, American Saddlebred: 42 horses, Morgan: 30 horses and Standardbred: 40 horses) were genotyped for the top SNP using StepOnePlus Real-Time PCR System (Life Technologies) with a custom TaqMan SNP genotyping assay (Applied Biosystems). A group of 49 Icelandic horses used for riding but that had not attended breeding field test was also genotyped. The sequence of the primers and probes was designed as follows: forward primer: 5′-GGAAGTTTCTAAACATTTTTGAAGGCTTTT-3′ (SEQ ID NO: 17); reverse primer: 5′-GGAGGGAAGTCAATTGACAAACG-3′ (SEQ ID NO: 18); mutant probe (FAM): 5′-CCTCCACGGCATCA-3′ (SEQ ID NO: 19); reference probe (VIC): 5′-TCCCTCCACAGCATCA-3′ (SEQ ID NO: 20). The reaction volume of 15 μl contained: 1.5 μl DNA, 0.38 μl Genotyping Assay 40X, 7.50 μl Genotyping Master Mix 2X, and 5.62 μl deionized water. The thermal cycle included 95° C. for 10 minutes, 40 cycles of 95° C. for 15 seconds, and 60° C. for 1 minute.

SNP genotyping of the DMRT3_Ser301STOP marker known as the “Gait Keeper” mutation was performed using custom designed TaqMan SNP Genotyping Assays (Applied Biosystem) as described previously (30,34).

Functional Annotation

The bioinformatics database NCBI was used to screen for candidate genes based on the EquCab3.0 reference genome and annotation release 103 (45) and HorseQTLdb release 41 to search for known quantitative trait loci (QTLs) for conformation in horses (40). Functional annotation of possible candidate genes was performed using the GeneCards database (46). All positions refer to the EquCab3.0 reference genome.

### Example 2

Materials and Methods

Two Icelandic horses with opposite haplotypes for the score of back and croup, were whole-genome sequenced (WGS) using Ilumina HiSeqX with paired-end 150 bp read length and 15× coverage. WGS analysis was performed according to GATK 3.8-0 Best Practices workflow (https://software.broadinstitute.org). The reads were aligned to the EquCab3.0 reference genome (GCF_002863925.1) using BWA-MEM 0.7.17 aligner. Variant calling was performed with HaplotypeCaller including a list of known variants in horses downloaded from NCBI webpage annotation release 103 (www.ncbi.nlm.nih.gov).

To compare the two horses, VCFtools 0.1.15 with the function diff-site was used. The identified QTL was extended and the investigated genomic region included ECA22: 45,200,000-45,800,000. WGS sequence data from 10 Icelandic horses were used to calculate LID between all SNPs within the region using VCFtools 0.1.15. Ensembl Variant Effect Predictor (VEP) (release 102) was used to investigate the effect of the variants.

Results

1148 SNPs and 95 indels were identified from the WGS analysis (Tables 8 and 9). A missense variant ECA22: 45,616,738 C/T (EquCab3) was identified within the serine/threonine-protein phosphatase 4 regulatory subunit 1 gene (LOC100056167). This missense variant was in complete LID (r²=1) with the top SNP from the genome-wide analysis.

TABLE 8
Indels identified from WGS analysis

| POS1 | POS2 | REF1 | REF2 | ALT1 | ALT2 |
|---|---|---|---|---|---|
| 45205733 | . | A | . | AGTGACTCTG | . |
| 45205830 | . | GGAAATGCTCCAA | . | G | . |
| 45208602 | . | G | . | GC | . |
| 45211273 | . | TC | . | T | . |
| 45215390 | . | G | . | GGTGT, GGTGTGT | . |
| 45238914 | . | T | . | TC | . |
| 45242471 | . | T | . | TG | . |
| 45246308 | . | C | . | CCCCA, CCCAA, CCA | . |
| 45247264 | . | T | . | TGCA | . |
| 45253103 | . | ACCGCC | . | A | . |
| 45253115 | . | CAATCATCACCAT | . | CCACCAT, * | . |
| 45253124 | . | CCAT | . | C, * | . |
| 45254483 | . | TC | . | TCC, TCCC, T | . |
| 45286120 | . | TG | . | T | . |
| 45310126 | . | AT | . | A, ATTTTT | . |
| 45319244 | . | TTTTGG | . | T | . |
| 45330489 | . | CATAG | . | C | . |
| 45335613 | . | GTGGA | . | G, GAGGAAGGATGGA | . |
| 45336672 | . | CAG | . | C | . |
| 45338556 | . | CCGGGTTCCAG | . | C | . |
| . | 45341927 | | GCCCACC | . | G, * |
| . | 45361572 | | GACACAC | . | G, GACAC, GAC |
| 45361601 | . | ACC | . | A | . |
| 45397376 | . | G | . | GA | . |
| 45399312 | . | TGG | . | TG, T | . |
| 45399774 | . | GAC | . | G | . |
| 45406126 | . | T | . | TG | . |
| 45411503 | . | TTTTC | . | T | . |
| 45422598 | . | TACCAAGGTAGTC | . | T | . |
| 45425722 | . | CGT | . | C | . |
| 45425725 | . | ATAGATAAAGCACAT | . | A | . |
| 45426686 | . | ATC | . | A | . |
| 45427793 | . | T | . | TG | . |
| 45432088 | . | GC | . | G | . |
| 45436192 | . | T | . | TA | . |
| 45443709 | . | A | . | AT | . |
| 45443838 | . | C | . | CT | . |
| 45445130 | . | G | . | GTT | . |
| 45450100 | . | GCCC | . | GCC, G, GC | . |
| 45450527 | . | CGG | . | C, * | . |
| 45450530 | . | ACAGCAGTTAAGTTT | . | A, * | . |
| | | GCGCGTTCTGCTTCA | | | |
| | | GGAGCCCGGGGTTC | | | |
| | | GAGGGTTCAGATCCC | | | |
| | | AGGTGCG | | | |
| 45467938 | . | C | | CA | . |
| 45467957 | . | CTTTT | . | C, CTTTTT | . |
| 45471799 | . | TG | . | T | . |
| 45471803 | . | TGTTTTTG | . | T | . |
| 45481832 | . | C | . | CA | . |
| 45483925 | . | CG | . | C | . |
| 45493330 | . | TG | . | TGGG, T | . |
| 45502347 | . | CA | . | CAAA, CAA, C | . |
| 45508390 | . | AT | . | A | . |
| 45508395 | . | A | . | AAAC | . |
| | 45516088 | . | GCCC | . | GCC, G, GC |
| 45529453 | . | AC | . | A | . |
| 45530858 | . | G | . | GGT | . |
| 45534123 | . | T | . | TA | . |
| . | 45537189 | . | C | . | CA |
| . | 45552622 | . | G | . | GT |
| . | 45556420 | . | ATT | . | A, ATTTTT, AT, |
| | | | | | ATTT, ATTTTTT |
| . | 45566412 | . | GTTT | . | GTTTT, GTT, G |
| . | 45573339 | . | CA | . | C |
| . | 45573808 | . | T | . | TG |
| . | 45576057 | . | G | . | GAAT |
| . | 45576196 | . | A | . | AG |
| . | 45576635 | . | TC | . | T |
| . | 45578066 | . | CG | . | C |
| . | 45578301 | . | AT | . | A |
| . | 45578358 | . | TTGTC | . | T |
| . | 45583829 | . | G | . | GATAACAAATGC |
| . | 45584767 | . | ACCC | . | A, ACC, AC |
| . | 45586812 | . | C | . | CT |
| . | 45590609 | . | CG | . | C |
| . | 45592068 | . | TCC | . | T |
| . | 45612646 | . | C | . | CA |
| . | 45612751 | . | T | . | TAGTC |
| 45621611 | . | T | | TC | . |
| | 45622460 | | CTG | . | C |
| 45628279 | . | TACAC | | TACACAC, T, | . |
| | | | | TACACACAC | |
| 45640449 | . | T | | TA | . |
| | 45645122 | | CAGGGAT | . | C |
| 45658346 | . | G | | GA | . |
| . | 45658504 | . | T | . | TC |
| . | 45660038 | . | CAA | . | C, CA |
| . | 45684956 | . | CT | . | C |
| . | 45694173 | . | GC | . | G, * |
| . | 45698201 | . | TA | . | T |
| . | 45708198 | . | GT | . | G |
| . | 45714389 | . | T | . | TC |
| 45726854 | | TA | . | T | . |
| . | 45736696 | . | AG | | A |
| . | 45761204 | . | T | . | TAGAAATGCA |
| 45768600 | . | ACT | . | A | . |
| 45774644 | . | G | . | GA | . |
| 45776104 | . | C | | CA | . |
| . | 45776145 | . | G | . | GGCCCC |
| 45789829 | | GT | . | G | . |

POS1 bp position within ECA22 of Icelandic horse with favorable haplotypes for the score of back and croup;
POS2 bp position within ECA22 of Icelandic horse with unfavorable haplotypes for the score of back and croup;
REF1 reference allele in Icelandic horse with favorable haplotypes for the score of back and croup;
REF2 reference allele in Icelandic horse with unfavorable haplotypes for the score of back and croup;
ALT1 alternate allele in Icelandic horse with favorable haplotypes for the score of back and croup;
ALT2 alternate allele in Icelandic horse with unfavorable haplotypes for the score of back and croup

TABLE 9
SNPs identified from WGS analysis

| POS | R1 | R2 | A1 | A2 | POS | R1 | R2 | A1 | A2 |
|---|---|---|---|---|---|---|---|---|---|
| 45200963 | C | · | T | · | 45469814 | C | · | G | · |
| 45201740 | A | · | G | · | 45470315 | A | · | G | · |
| 45201861 | A | · | T | · | 45470633 | G | · | A | · |
| 45203574 | T | · | C | · | 45470737 | C | · | T | · |
| 45203829 | C | · | T | · | 45470819 | A | · | G | · |
| 45204618 | G | · | A | · | 45470864 | G | · | A | · |
| 45205014 | G | · | A | · | 45471058 | A | · | C | · |
| 45205612 | T | · | C | · | 45471076 | T | · | G | · |
| 45205910 | T | · | C | · | 45471107 | T | · | C | · |
| 45207626 | G | · | T | · | 45471114 | G | · | C | · |
| 45207800 | T | · | A | · | 45471171 | C | · | T | · |
| 45208030 | T | · | C | · | 45471227 | A | · | G | · |
| 45208348 | T | · | C | · | 45471273 | G | · | A | · |
| 45208709 | T | · | C | · | 45471357 | A | · | C | · |
| 45209172 | G | · | T | · | 45471396 | A | · | C | · |
| 45211077 | C | · | G | · | 45471397 | T | · | C | · |
| 45214612 | C | · | T | · | 45471422 | T | · | C | · |
| 45216096 | A | · | G | · | 45471450 | C | · | T | · |
| 45217847 | T | · | C | · | 45471451 | A | · | G | · |
| 45218067 | T | · | C | · | 45471530 | A | · | C | · |
| 45218265 | G | · | T | · | 45471586 | A | · | G | · |
| 45218283 | T | · | G | · | 45471598 | T | · | G | · |
| 45219655 | C | · | G | · | 45471623 | G | · | A | · |
| 45219806 | C | · | T | · | 45471639 | G | · | A | · |
| 45220230 | G | · | A | · | 45471663 | A | · | G | · |
| 45220543 | G | · | A | · | 45471711 | C | · | T | · |
| 45220882 | A | · | G | · | 45471723 | C | · | T | · |
| 45221505 | T | · | C | · | 45471809 | T | · | G, * | · |
| 45221535 | A | · | G | · | 45471815 | G | · | T | · |
| 45222034 | C | · | T | · | 45472559 | C | · | T | · |
| 45222613 | A | · | C | · | 45472829 | G | · | A | · |
| 45223820 | T | · | C | · | 45472861 | C | · | T | · |
| 45224287 | C | · | T | · | 45472915 | A | · | G | · |
| 45224340 | A | · | G | · | 45473520 | T | · | C | · |
| 45224522 | G | · | A | · | 45474116 | G | · | A | · |
| 45224634 | G | · | C | · | 45474346 | C | · | T | · |
| 45225285 | G | · | T | · | 45474547 | A | · | G | · |
| 45226332 | G | · | A | · | 45474577 | G | · | A | · |
| 45226654 | C | · | T | · | 45474931 | T | · | C | · |
| 45226838 | C | · | T | · | 45475236 | T | · | C | · |
| 45227596 | C | · | G | · | 45475267 | T | · | C | · |
| 45229063 | C | · | A | · | 45475488 | T | · | C | · |
| 45229275 | A | · | G | · | 45475756 | A | · | T | · |
| 45229604 | G | · | A | · | 45475938 | C | · | T | · |
| 45230658 | T | · | C | · | 45476500 | T | · | C | · |
| 45231356 | T | · | C | · | 45476754 | G | · | A | · |
| 45231445 | C | · | T | · | 45476946 | C | · | A | · |
| 45231788 | G | · | A | · | 45476958 | A | · | G | · |
| 45231800 | G | · | A | · | 45478691 | C | · | T | · |
| 45231924 | T | · | C | · | 45479189 | C | · | T | · |
| 45232119 | C | · | A | · | 45479768 | C | · | T | · |
| 45233418 | G | · | A | · | 45479925 | C | · | T | · |
| 45234257 | T | · | G | · | 45480227 | C | · | A | · |
| 45234576 | G | · | T | · | 45480782 | C | · | T | · |
| 45234719 | A | · | G | · | 45480852 | A | · | G | · |
| 45234906 | G | · | T | · | 45480892 | · | · | · | G |
| 45235035 | T | · | C | · | 45480898 | · | · | · | A |
| 45235318 | G | · | A | · | 45480925 | · | · | · | T |
| 45236767 | A | · | G | · | 45480926 | · | · | · | A |
| 45237626 | C | · | T | · | 45480929 | · | · | · | C |
| 45238199 | T | · | C, * | · | 45480935 | · | · | · | T, C |
| 45239046 | G | · | T | · | 45480958 | · | · | · | C |
| 45239700 | G | · | C | · | 45481027 | · | · | · | C |
| 45240483 | C | · | G | · | 45481031 | · | · | · | G |
| 45240532 | C | · | T | · | 45481032 | · | · | · | G |
| 45240954 | G | · | A | · | 45481055 | · | · | · | A |
| 45241502 | T | · | C | · | 45481057 | · | · | · | A |
| 45241717 | C | · | T | · | 45481060 | · | · | · | C |
| 45241886 | T | · | C | · | 45481079 | · | · | · | A |
| 45242702 | T | · | C | · | 45481091 | · | · | · | A |
| 45243156 | G | · | A | · | 45481099 | · | · | · | A |
| 45243978 | A | · | G | · | 45481952 | A | · | G | · |
| 45244552 | G | · | A | · | 45482679 | G | · | A | · |
| 45244738 | G | · | A | · | 45483107 | A | · | G | · |
| 45245390 | A | · | G | · | 45483926 | G | · | A, * | · |
| 45246147 | G | · | A | · | 45483927 | G | · | A | · |
| 45246788 | C | · | T | · | 45484495 | A | · | G | · |
| 45246842 | T | · | C | · | 45484496 | C | · | G | · |
| 45246865 | C | · | A | · | 45484794 | C | · | T | · |
| 45247313 | G | · | C | · | 45484896 | T | · | G | · |
| 45247314 | T | · | C | · | 45485474 | C | · | A | · |
| 45247847 | T | · | C | · | 45486009 | C | · | T | · |
| 45247884 | C | · | T | · | 45486500 | G | · | A | · |
| 45253106 | G | · | A, * | · | 45487262 | A | · | G | · |
| 45255317 | G | · | T | · | 45487428 | G | · | C | · |
| 45255854 | G | · | A | · | 45488658 | G | · | A | · |
| 45256142 | T | · | C | · | 45488982 | G | · | C | · |
| 45257445 | C | · | T | · | 45489648 | C | · | T | · |
| 45258951 | T | · | C | · | 45491014 | T | · | C | · |
| 45264119 | T | · | C | · | 45491829 | T | · | C | · |
| 45265301 | C | · | T | · | 45493064 | C | · | G, A | · |
| 45267599 | T | · | C | · | 45493097 | T | · | G | · |
| 45269083 | A | · | G | · | 45493141 | A | · | G | · |
| 45270793 | T | · | C | · | 45494106 | C | · | G | · |
| 45270851 | G | · | A | · | 45494455 | T | · | G | · |
| 45271973 | G | · | A | · | 45494685 | T | · | A | · |
| 45273218 | C | · | T | · | 45495197 | C | · | G, A | · |
| 45274713 | G | · | A | · | 45495723 | A | · | C | · |
| 45277197 | T | · | C | · | 45495899 | C | · | T | · |
| 45277277 | A | · | G | · | 45497594 | A | · | G | · |
| 45277360 | T | · | A, * | · | 45497771 | G | · | A | · |
| 45277621 | A | · | G | · | 45497961 | T | · | C | · |
| 45277980 | G | · | A | · | 45498296 | C | · | T | · |
| 45278009 | C | · | T | · | 45498834 | G | · | A | · |
| 45278286 | G | · | A | · | 45498857 | T | · | C | · |
| 45278633 | T | · | A, C | · | 45498891 | T | · | C | · |
| 45278782 | T | · | G | · | 45498899 | A | · | G | · |
| 45278819 | T | · | C | · | 45499282 | G | · | A | · |
| 45279117 | T | · | C | · | 45499523 | T | · | C | · |
| 45279682 | T | · | C | · | 45499605 | G | · | T | · |
| 45279724 | C | · | A | · | 45499858 | G | · | C | · |
| 45281404 | A | · | G | · | 45499998 | T | · | C | · |
| 45281662 | G | · | A | · | 45500011 | T | · | A | · |
| 45281729 | G | · | A | · | 45500171 | T | · | C | · |
| 45283377 | T | · | C | · | 45500229 | T | · | A | · |
| 45283655 | A | · | G | · | 45500282 | C | · | T | · |
| 45283715 | C | · | T | · | 45500286 | A | · | G | · |
| 45283927 | C | · | T | · | 45500367 | A | · | G | · |
| 45284129 | C | · | T | · | 45500466 | A | · | G | · |
| 45284398 | T | · | C | · | 45500494 | A | · | G | · |
| 45284966 | T | · | G | · | 45500718 | C | · | G | · |
| 45286647 | T | · | A | · | 45500923 | G | · | C | · |
| 45288023 | C | · | T | · | 45501246 | C | · | A | · |
| 45288044 | C | · | T | · | 45501335 | A | · | G | · |
| 45288393 | A | · | G | · | 45501459 | A | · | G | · |
| 45288997 | T | · | C | · | 45501941 | G | · | A | · |
| 45289156 | C | · | A | · | 45502400 | T | · | C | · |
| 45290421 | T | · | C | · | 45502411 | G | · | T | · |
| 45290591 | C | · | A | · | 45502582 | A | · | C | · |
| 45290837 | T | · | A | · | 45502598 | A | · | G | · |
| 45291106 | T | · | G | · | 45503097 | C | · | T | · |
| 45291382 | C | · | T | · | 45503361 | T | · | C | · |
| 45293990 | A | · | G | · | 45503791 | C | · | T | · |
| 45294056 | G | · | A | · | 45503801 | A | · | G | · |
| 45294319 | G | · | C | · | 45506794 | A | · | G | · |
| 45295103 | T | · | C | · | 45508391 | T | · | A, * | · |
| 45296978 | T | · | C | · | 45508392 | G | · | A | · |
| 45297246 | T | · | C | · | 45508477 | A | · | G | · |
| 45297294 | C | · | T | · | 45509832 | T | · | C | · |
| 45298646 | G | · | A | · | 45510948 | T | · | C | · |
| 45298772 | G | · | A | · | 45511768 | G | · | A | · |
| 45299219 | C | · | T | · | 45512141 | G | · | A | · |
| 45299421 | G | · | A | · | 45512184 | C | · | A | · |
| 45300620 | C | · | T | · | 45512186 | G | · | A | · |
| 45301064 | A | · | G | · | 45512838 | C | · | T | · |
| 45301160 | G | · | A | · | 45514038 | T | · | C | · |
| 45301228 | G | · | A | · | 45515360 | C | · | G | · |
| 45303514 | C | · | T, G | · | 45515643 | C | · | T | · |
| 45303671 | A | · | G | · | 45516119 | T | · | C | · |
| 45304284 | T | · | C | · | 45516595 | A | · | G | · |
| 45304707 | T | · | C | · | 45516801 | A | · | T | · |
| 45304903 | G | · | T | · | 45516897 | T | · | C | · |
| 45305268 | C | · | T | · | 45517460 | G | · | A | · |
| 45305466 | C | · | A | · | 45518951 | G | · | T | · |
| 45306812 | A | · | G | · | 45519505 | C | · | T | · |
| 45308538 | C | · | T | · | 45520139 | G | · | A | · |
| 45308719 | A | · | G | · | 45521183 | A | · | G | · |
| 45308744 | A | · | G | · | 45524597 | T | · | G | · |
| 45309963 | G | · | A | · | 45524711 | T | · | C | · |
| 45310127 | T | · | C, * | · | 45524731 | T | · | C | · |
| 45312076 | G | · | A | · | 45525842 | G | · | A | · |
| 45312826 | T | · | C | · | 45526050 | A | · | G | · |
| 45314307 | C | · | T | · | 45526577 | G | · | A | · |
| 45314580 | G | · | A | · | 45526608 | C | · | T | · |
| 45315926 | T | · | C | · | 45526687 | T | · | C | · |
| 45316823 | C | · | T | · | 45527436 | T | · | C | · |
| 45317189 | A | · | G | · | 45527438 | C | · | A, G | · |
| 45319520 | C | · | A | · | 45527709 | G | · | A | · |
| 45319596 | C | · | T | · | 45527759 | G | · | A | · |
| 45319842 | A | · | G | · | 45527802 | G | · | A | · |
| 45321137 | G | · | A | · | 45529414 | T | · | G | · |
| 45322538 | G | · | A | · | 45529450 | A | · | G | · |
| 45323415 | C | · | T | · | 45529521 | A | · | G | · |
| 45323918 | T | · | G | · | 45529531 | C | · | G | · |
| 45324695 | A | · | G | · | 45529534 | T | · | A | · |
| 45324908 | C | · | T | · | 45529541 | A | · | G | · |
| 45325165 | A | · | G | · | 45529581 | T | · | C | · |
| 45325896 | A | · | G | · | 45529585 | A | · | C | · |
| 45326006 | G | · | A | · | 45529667 | C | · | T | · |
| 45327715 | G | · | A | · | 45529715 | A | · | G | · |
| 45329104 | A | · | G | · | 45529732 | A | · | G | · |
| 45329347 | G | · | A | · | 45529777 | T | · | C | · |
| 45329916 | G | · | A | · | 45529789 | T | · | C | · |
| 45329984 | C | · | T | · | 45529790 | G | · | A | · |
| 45330011 | T | · | C | · | 45529833 | A | · | G | · |
| 45330028 | C | · | T | · | 45529879 | C | · | G | · |
| 45330065 | A | · | G | · | 45529917 | T | · | C | · |
| 45330138 | C | · | T | · | 45529918 | A | · | G | · |
| 45330181 | G | · | T | · | 45529940 | A | · | G | · |
| 45330586 | A | · | G | · | 45529948 | T | · | A, G | · |
| 45330968 | G | · | A | · | 45530035 | T | · | C | · |
| 45332063 | T | · | C | · | 45530087 | A | · | G | · |
| 45332350 | G | · | A | · | 45530216 | A | · | G, * | · |
| 45332352 | G | · | A | · | 45530217 | T | · | C, * | · |
| 45332620 | C | · | G | · | 45530262 | T | · | C | · |
| 45332810 | T | · | C | · | 45530297 | A | · | C | · |
| 45335963 | T | · | C | · | 45530359 | C | · | T | · |
| 45336695 | A | · | G | · | 45530360 | A | · | G | · |
| 45336734 | G | · | A | · | 45530371 | A | · | G | · |
| 45336751 | A | · | G | · | 45530372 | T | · | C | · |
| 45336791 | A | · | G | · | 45530378 | G | · | A | · |
| 45336822 | C | · | T | · | 45530571 | A | · | G | · |
| 45336954 | G | · | T | · | 45530700 | C | · | T | · |
| 45336983 | T | · | C | · | 45530790 | C | · | T | · |
| 45337088 | C | · | T | · | 45530814 | C | · | T | · |
| 45337245 | G | · | A | · | 45530819 | A | · | G | · |
| 45337255 | C | · | G | · | 45530843 | G | · | A | · |
| 45337281 | C | · | T | · | 45530870 | C | · | T | · |
| 45337306 | T | · | C | · | 45530888 | T | · | C | · |
| 45337333 | T | · | C | · | 45530931 | G | · | A | · |
| 45337355 | T | · | G | · | 45530959 | T | · | C | · |
| 45337363 | T | · | C | · | 45530998 | G | · | A | · |
| 45337487 | C | · | A | · | 45531104 | T | · | G | · |
| 45337501 | T | · | G | · | 45531118 | A | · | T | · |
| 45337624 | G | · | A | · | 45531332 | C | · | T | · |
| 45337625 | A | · | G | · | 45532931 | A | · | G | · |
| 45337629 | T | · | A | · | 45533130 | A | · | T | · |
| 45337721 | G | · | A | · | 45535124 | C | · | T | · |
| 45337887 | G | · | A | · | 45537305 | G | · | A | · |
| 45337960 | C | · | G | · | 45537717 | · | G | · | T |
| 45338067 | G | · | A | · | 45538217 | · | G | · | A |
| 45338186 | C | · | T | · | 45539128 | A | · | G | · |
| 45338339 | G | · | C | · | 45539281 | T | · | C | · |
| 45338357 | G | · | T | · | 45539449 | · | C | · | A |
| 45338368 | C | · | G | · | 45540067 | G | · | A | · |
| 45338514 | G | · | A | · | 45540097 | G | · | T | · |
| 45338534 | T | · | C | · | 45540652 | · | T | · | C |
| 45338537 | A | · | G | · | 45541664 | · | G | · | A |
| 45338573 | T | · | C | · | 45542631 | · | A | · | G |
| 45338586 | A | · | G | · | 45542829 | · | A | · | G |
| 45338658 | T | · | C | · | 45543031 | · | G | · | A |
| 45338659 | G | · | A | · | 45543254 | · | A | · | G |
| 45338698 | A | · | G | · | 45543899 | · | A | · | G |
| 45338859 | G | · | A | · | 45544298 | · | A | · | G |
| 45338994 | A | · | G | · | 45544492 | · | T | · | A, * |
| 45339033 | A | · | G | · | 45547002 | · | G | · | A |
| 45339299 | C | · | A, T | · | 45548189 | · | G | · | T |
| 45339340 | G | · | A | · | 45548271 | · | C | · | A |
| 45339342 | G | · | A | · | 45553372 | · | A | · | G |
| 45339357 | T | · | C | · | 45553921 | · | A | · | G |
| 45339370 | C | · | T | · | 45555898 | · | A | · | C |
| 45339376 | A | · | G | · | 45556130 | · | T | · | C |
| 45339816 | G | · | A | · | 45557129 | C | · | T | · |
| 45340532 | G | · | A | · | 45557347 | · | T | · | C |
| 45341040 | A | · | G | · | 45559144 | · | T | · | C |
| 45341190 | G | · | A | · | 45559178 | · | T | · | C |
| 45341219 | T | · | G | · | 45560888 | · | A | · | G |
| 45342144 | · | T | · | C | 45561720 | · | G | · | A |
| 45342842 | C | · | T | · | 45562563 | · | G | · | A |
| 45343296 | · | C | · | T | 45565664 | · | T | · | C |
| 45343520 | · | C | · | A | 45566583 | · | T | · | C |
| 45343524 | C | · | T | · | 45569847 | · | C | · | T |
| 45344392 | · | C | · | T | 45570911 | · | A | · | C |
| 45344608 | G | · | A | · | 45572115 | · | G | · | A |
| 45346235 | · | G | · | A | 45572674 | · | G | · | A |
| 45347373 | C | · | G | · | 45573355 | · | C | · | T |
| 45347522 | · | T | · | C | 45573746 | · | C | · | T |
| 45348095 | G | · | C | · | 45573785 | · | G | · | A |
| 45350158 | · | C | · | T | 45573957 | · | G | · | A |
| 45350172 | C | · | T | · | 45573974 | · | T | · | C |
| 45350244 | G | · | A | · | 45573976 | · | T | · | C |
| 45351566 | C | · | T | · | 45573987 | · | T | · | C |
| 45351807 | C | · | T, A | · | 45574069 | · | G | · | A |
| 45351988 | · | G | · | A | 45574093 | · | C | · | A |
| 45352007 | G | · | A | · | 45574094 | · | T | · | G |
| 45352028 | T | · | G | · | 45574172 | · | C | · | G |
| 45352477 | T | · | C | · | 45574223 | · | T | · | G |
| 45353338 | · | A | · | T | 45574305 | · | C | · | A |
| 45354290 | · | C | · | T | 45574333 | · | T | · | C |
| 45354912 | · | G | · | A | 45574408 | · | T | · | C |
| 45354964 | · | C | · | T | 45574542 | · | G | · | A |
| 45356792 | · | G | · | A | 45574583 | · | T | · | C |
| 45357456 | C | · | T | · | 45574595 | · | T | · | G |
| 45358695 | · | G | · | C | 45574693 | · | T | · | C |
| 45360208 | · | A | · | G | 45574716 | · | C | · | T |
| 45360573 | A | · | C | · | 45574753 | · | C | · | T |
| 45360672 | C | · | T | · | 45574762 | · | T | · | C |
| 45360714 | T | · | C | · | 45574814 | · | G | · | A |
| 45361603 | C | · | A, * | · | 45574841 | · | T | · | C |
| · | · | C | · | T | 45575000 | · | A | · | G |
| · | · | T | · | G | 45575233 | · | C | · | T |
| · | · | C | · | A | 45575244 | · | A | · | G |
| 45363022 | G | · | A | · | 45575312 | · | C | · | G |
| · | · | A | · | C | 45575422 | · | G | · | T |
| · | · | G | · | A | 45575651 | · | T | · | C |
| 45367115 | C | · | T | · | 45575652 | · | G | · | A |
| 45367132 | T | · | C | · | 45575765 | · | G | · | A |
| · | · | C | · | G | 45575785 | · | G | · | A |
| · | · | T | · | G, A | 45575817 | · | T | · | G |
| · | · | T | · | C | 45575825 | · | T | · | C |
| 45369966 | G | · | A | · | 45575837 | · | G | · | A |
| 45378667 | C | · | A | · | 45575936 | · | C | · | G |
| 45379461 | A | · | G | · | 45576106 | · | A | · | T |
| 45379519 | A | · | G | · | 45576107 | · | G | · | A |
| 45379942 | G | · | A | · | 45576124 | · | G | · | A |
| 45380449 | T | · | C | · | 45576137 | · | G | · | A |
| 45380701 | A | · | C | · | 45576152 | · | C | · | T |
| 45384863 | A | · | G | · | 45576200 | · | T | · | A |
| 45385146 | A | · | G | · | 45576236 | · | C | · | T |
| 45385253 | C | · | T | · | 45576269 | · | C | · | T |
| 45385539 | T | · | C | · | 45576284 | · | C | · | T |
| 45386037 | T | · | C | · | 45576312 | · | T | · | C |
| 45386068 | C | · | T | · | 45576472 | · | A | · | G |
| 45388198 | A | · | G | · | 45576477 | · | G | · | A |
| 45388495 | T | · | C | · | 45576624 | · | C | · | T |
| 45389428 | C | · | G | · | 45576713 | · | C | · | A |
| 45390491 | C | · | T | · | 45576720 | · | T | · | C |
| 45392021 | A | · | T | · | 45576728 | · | A | · | G |
| 45392143 | G | · | A | · | 45576748 | · | T | · | C |
| 45393383 | G | · | A | · | 45576903 | · | G | · | A |
| 45394983 | T | · | C | · | 45576991 | · | G | · | A |
| 45395058 | C | · | T | · | 45577191 | · | A | · | G |
| 45395107 | C | · | A | · | 45577911 | · | C | · | A |
| 45395512 | C | · | T | · | 45577912 | · | C | · | T |
| 45395637 | T | · | C | · | 45578536 | · | T | · | C |
| 45395943 | A | · | G | · | 45578811 | · | C | · | T |
| 45396228 | A | · | G | · | 45578895 | · | G | · | A |
| 45396452 | T | · | C | · | 45579012 | · | T | · | C |
| 45396929 | A | · | G | · | 45579220 | · | G | · | A |
| 45397510 | C | · | T | · | 45579283 | · | C | · | T, * |
| 45398742 | G | · | T | · | 45579546 | · | A | · | G |
| 45400621 | G | · | C | · | 45579844 | · | C | · | T |
| 45401658 | G | · | C | · | 45579848 | · | A | · | G |
| 45401941 | A | · | G | · | 45580084 | · | G | · | A |
| 45402477 | C | · | T | · | 45580349 | · | C | · | T |
| 45402697 | G | · | A | · | 45580378 | · | A | · | G |
| 45403903 | T | · | G | · | 45581541 | · | G | · | A |
| 45404120 | C | · | T | · | 45581549 | · | C | · | T |
| 45404138 | A | · | G | · | 45582233 | · | A | · | G |
| 45404217 | G | · | A, T | · | 45582577 | · | T | · | G |
| 45404934 | A | · | G | · | 45582645 | · | G | · | A |
| 45407620 | C | · | T | · | 45582705 | · | C | · | G |
| 45408232 | T | · | C | · | 45582711 | · | T | · | C |
| 45408694 | C | · | T | · | 45582750 | · | T | · | C |
| 45408767 | A | · | G | · | 45582763 | · | G | · | T |
| 45410034 | A | · | G | · | 45583300 | · | T | · | C |
| 45410062 | A | · | G | · | 45583646 | · | C | · | T |
| 45411067 | G | · | C | · | 45583685 | · | G | · | A |
| 45411817 | G | · | A | · | 45583740 | · | A | · | G |
| 45412089 | G | · | A | · | 45583784 | · | A | · | G |
| 45412337 | A | · | G | · | 45584617 | · | A | · | C |
| 45412841 | C | · | T | · | 45585294 | · | A | · | T |
| 45413294 | T | · | C | · | 45585408 | · | C | · | T |
| 45414283 | G | · | A | · | 45585440 | · | A | · | G |
| 45414405 | C | · | T | · | 45585573 | · | T | · | G |
| 45414497 | T | · | C | · | 45585620 | · | G | · | A |
| 45414594 | T | · | C | · | 45585635 | · | G | · | A |
| 45415796 | G | · | A | · | 45585682 | · | G | · | A |
| 45415828 | G | · | A | · | 45585689 | · | A | · | G |
| 45416535 | A | · | G | · | 45585885 | · | T | · | C |
| 45416605 | C | · | T | · | 45585886 | · | G | · | A |
| 45417737 | G | · | C | · | 45586010 | · | G | · | A |
| 45417815 | A | · | G | · | 45586808 | · | T | · | A |
| 45418428 | T | · | C | · | 45586986 | · | C | · | T, G |
| 45418556 | G | · | A | · | 45587769 | · | T | · | A |
| 45418654 | A | · | G | · | 45588027 | · | T | · | A |
| 45419397 | T | · | C | · | 45588028 | · | T | · | A |
| 45419637 | T | · | C | · | 45588412 | · | G | · | A |
| 45420066 | A | · | G | · | 45588535 | · | G | · | C |
| 45420074 | C | · | T | · | 45588603 | · | G | · | T |
| 45420285 | G | · | A | · | 45588657 | · | G | · | C |
| 45421254 | G | · | A | · | 45588785 | · | C | · | T |
| 45421416 | G | · | A | · | 45588810 | · | G | · | A |
| 45421588 | G | · | A | · | 45588844 | · | T | · | C |
| 45422014 | A | · | G | · | 45588849 | · | T | · | C |
| 45422042 | G | · | A | · | 45588889 | · | G | · | A |
| 45422326 | G | · | A | · | 45588964 | · | G | · | T |
| 45422757 | G | · | A | · | 45589156 | · | C | · | T |
| 45422842 | T | · | C | · | 45589199 | · | C | · | A |
| 45422872 | C | · | A | · | 45589922 | · | A | · | T |
| 45422956 | C | · | T | · | 45589970 | · | T | · | C |
| 45423047 | C | · | T | · | 45590202 | · | G | · | A |
| 45423076 | G | · | A | · | 45590258 | · | G | · | A |
| 45423116 | C | · | T | · | 45590400 | · | T | · | G |
| 45423185 | A | · | C | · | 45590431 | · | G | · | A,C |
| 45423280 | C | · | T | · | 45590566 | · | G | · | A |
| 45423291 | C | · | T | · | 45590569 | · | A | · | G |
| 45423432 | G | · | A | · | 45590610 | · | G | · | C .* |
| 45423671 | G | · | A | · | 45590728 | · | A | · | G |
| 45423869 | A | · | G | · | 45590993 | · | G | · | A |
| 45424012 | G | · | A | · | 45591126 | · | C | · | T |
| 45424075 | A | · | G | · | 45591533 | · | A | · | G |
| 45424080 | T | · | C | · | 45592441 | · | G | · | A |
| 45424123 | T | · | G | · | 45594288 | · | T | · | C |
| 45424302 | T | · | C | · | 45595325 | · | G | · | A |
| 45424387 | T | · | C | · | 45595498 | · | G | · | T,* |
| 45424537 | G | · | C | · | 45595731 | · | G | · | A |
| 45424542 | A | · | G | · | 45595769 | · | C | · | T |
| 45424685 | G | · | A | · | 45596241 | · | T | · | C |
| 45424712 | T | · | C | · | 45597711 | · | T | · | C |
| 45425007 | C | · | T | · | 45599481 | · | C | · | T |
| 45425157 | G | · | A | · | 45599487 | · | T | · | C |
| 45425296 | G | · | A | · | 45601506 | · | A | · | G |
| 45425349 | A | · | T | · | 45602222 | · | G | · | A |
| 45425352 | C | · | G | · | 45602937 | · | A | · | G |
| 45425373 | A | · | C | · | 45604315 | · | G | · | A |
| 45425601 | A | · | G | · | 45604373 | · | G | · | C |
| 45425634 | C | · | T | · | 45605284 | · | A | · | G |
| 45425781 | C | · | T | · | 45606784 | · | G | · | A |
| 45425790 | T | · | C | · | 45607049 | · | G | · | A |
| 45425854 | T | · | C | · | 45610157 | A | · | G | · |
| 45425865 | T | · | C | · | 45613656 | · | A | · | G |
| 45425895 | A | · | G | · | 45614363 | · | G | · | A |
| 45425975 | G | · | A | · | 45615584 | T | · | C | · |
| 45426097 | T | · | G | · | 45616738 | C | · | T | · |
| 45426268 | C | · | T | · | 45617234 | · | G | · | A |
| 45426318 | G | · | A | · | 45617653 | T | · | C | · |
| 45426404 | A | · | G | · | 45620791 | A | · | T | · |
| 45426477 | C | · | T | · | 45621113 | C | · | A | · |
| 45426527 | T | · | C | · | 45621488 | T | · | G | · |
| 45426678 | C | · | G | · | 45622129 | A | · | G | · |
| 45426767 | T | · | C | · | 45622744 | A | · | G | · |
| 45426946 | T | · | C | · | 45624382 | · | G | · | A |
| 45427126 | C | · | A | · | 45626415 | · | G | · | A |
| 45427169 | C | · | T | · | 45626541 | G | · | C | · |
| 45427193 | G | · | A | · | 45626709 | T | · | G | · |
| 45427300 | G | · | C | · | 45627891 | · | C | · | A |
| 45427333 | C | · | G | · | 45632175 | · | C | · | T |
| 45427481 | G | · | A | · | 45633615 | · | T | · | C |
| 45427616 | C | · | T | · | 45634462 | C | · | T | · |
| 45427846 | A | · | G | · | 45635175 | · | C | · | T |
| 45427897 | T | · | C | · | 45636293 | · | G | · | A |
| 45428170 | G | · | A | · | 45636607 | A | · | G | · |
| 45428209 | G | · | A | · | 45636748 | A | · | C | · |
| 45428289 | C | · | T | · | 45637135 | T | · | A | · |
| 45428805 | A | · | G | · | 45637234 | C | · | G | · |
| 45428828 | G | · | A | · | 45642393 | · | A | · | C |
| 45429119 | C | · | T | · | 45643145 | G | · | C | · |
| 45429684 | C | · | T | · | 45643490 | A | · | C | · |
| 45429689 | G | · | A | · | 45646210 | · | C | · | T |
| 45429730 | C | · | T | · | 45646598 | T | · | C | · |
| 45430200 | G | · | A | · | 45647253 | T | · | C | · |
| 45430341 | T | · | C | · | 45648952 | T | · | A | · |
| 45430367 | T | · | C | · | 45649277 | C | · | G | · |
| 45430378 | A | · | G | · | 45649689 | · | C | · | T |
| 45430734 | A | · | G | · | 45651161 | A | · | G | · |
| 45430747 | C | · | T | · | 45652628 | C | · | T | · |
| 45430838 | G | · | A | · | 45653086 | · | G | · | A |
| 45430861 | A | · | G | · | 45653096 | A | · | T | · |
| 45430914 | A | · | G | · | 45653381 | · | A | · | C |
| 45430930 | A | · | G | · | 45654309 | C | · | T | · |
| 45431015 | C | · | T | · | 45655812 | T | · | C | · |
| 45431068 | T | · | A | · | 45655982 | · | A | · | T |
| 45431145 | C | · | G | · | 45656229 | A | · | C | · |
| 45431420 | A | · | G | · | 45656281 | G | · | C | · |
| 45431710 | A | · | G | · | 45656707 | · | C | · | T |
| 45431714 | T | · | C | · | 45657231 | · | G | · | A |
| 45431788 | G | · | A | · | 45658011 | · | C | · | T |
| 45431813 | G | · | C | · | 45658333 | T | · | C | · |
| 45431924 | C | · | A | · | 45658737 | T | · | C | · |
| 45431944 | G | · | A | · | 45658882 | G | · | A | · |
| 45432157 | G | · | C | · | 45659319 | · | G | · | A |
| 45432176 | C | · | T | · | 45659605 | · | G | · | C |
| 45432244 | C | · | A | · | 45659934 | A | · | G | · |
| 45432519 | T | · | G | · | 45660039 | · | A | · | C, * |
| 45432650 | C | · | A | · | 45660159 | · | A | · | C |
| 45432659 | G | · | A | · | 45662708 | T | · | C | · |
| 45433089 | C | · | T | · | 45664010 | · | A | · | G |
| 45433098 | T | · | C | · | 45664863 | C | · | T | · |
| 45433144 | G | · | A | · | 45665875 | G | · | A | · |
| 45433147 | G | · | A | · | 45666605 | · | T | · | G |
| 45433190 | C | · | T | · | 45667672 | G | · | A | · |
| 45433191 | T | · | G | · | 45668023 | A | · | G | · |
| 45433293 | T | · | C | · | 45669386 | · | T | · | A |
| 45433384 | C | · | T | · | 45669504 | · | C | · | T |
| 45433747 | G | · | A | · | 45670970 | A | · | G | · |
| 45433748 | T | · | A, C | · | 45673400 | G | · | A | · |
| 45433939 | C | · | A | · | 45673404 | A | · | G | · |
| 45433996 | A | · | C | · | 45674485 | A | · | C | · |
| 45433997 | G | · | A | · | 45674653 | G | · | T | · |
| 45434012 | A | · | G | · | 45677791 | · | A | · | C |
| 45434309 | T | · | C | · | 45677889 | C | · | T | · |
| 45434484 | T | · | C | · | 45678151 | G | · | A | · |
| 45434485 | G | · | A | · | 45678411 | T | · | C | · |
| 45434541 | C | · | T | · | 45679337 | C | · | T | · |
| 45434649 | C | · | T | · | 45679711 | T | · | C | · |
| 45434724 | G | · | A | · | 45681196 | · | T | · | A |
| 45434753 | G | · | C | · | 45682390 | C | · | T | · |
| 45434814 | C | · | A | · | 45682573 | T | · | G | · |
| 45434822 | C | · | T | · | 45683106 | · | T | · | C |
| 45434885 | T | · | C | · | 45683693 | · | A | · | C |
| 45435225 | C | · | G | · | 45684902 | G | . | A | · |
| 45435700 | T | · | C | · | 45687616 | G | . | A | · |
| 45435704 | C | · | T | · | 45689184 | T | . | A | · |
| 45435705 | A | · | G | · | 45689243 | T | · | C | · |
| 45435748 | C | · | T | · | 45689608 | · | C | · | G |
| 45436327 | C | · | G, T | · | 45689938 | · | T | · | G |
| 45436374 | T | · | C | · | 45692566 | C | · | G | · |
| 45436800 | T | · | C | · | 45692574 | C | · | G | · |
| 45437867 | T | · | C | · | 45692579 | A | · | G | · |
| 45439766 | A | · | G | · | 45692581 | T | · | G | · |
| 45441708 | G | · | A | · | 45692586 | A | · | C | · |
| 45442810 | C | · | T | · | 45692824 | · | G | · | A |
| 45443000 | G | · | A | · | 45692983 | · | G | · | T |
| 45443139 | T | · | C | · | 45695144 | · | G | · | A |
| 45443296 | C | · | T | · | 45698942 | A | · | G | · |
| 45443577 | C | · | T | · | 45701197 | · | T | · | A |
| 45443608 | C | · | T | · | 45702813 | · | A | · | T |
| 45443842 | C | · | A | · | 45702879 | · | G | · | A |
| 45444173 | C | · | T | · | 45704045 | A | · | G | · |
| 45444211 | C | · | A | · | 45705467 | · | C | · | T |
| 45444212 | A | · | G | · | 45705794 | C | · | T | · |
| 45444216 | C | · | T | · | 45707143 | · | A | · | G |
| 45444344 | C | · | T | · | 45708200 | · | T | · | C |
| 45444430 | C | · | T | · | 45710533 | T | · | C | · |
| 45444778 | A | · | G | · | 45711375 | T | · | C | · |
| 45444795 | T | · | A | · | 45711864 | · | T | · | C |
| 45445006 | A | · | G | · | 45712418 | · | G | · | A |
| 45445066 | G | · | A | · | 45715897 | G | · | A | · |
| 45445071 | A | · | G | · | 45721786 | · | C | · | T |
| 45445111 | A | · | G | · | 45723722 | C | · | T | · |
| 45445112 | A | · | C | · | 45723980 | C | · | T | · |
| 45445127 | G | · | T | · | 45723983 | A | · | G | · |
| 45445350 | C | · | T | · | 45728583 | · | C | · | T |
| 45445429 | A | · | G | · | 45730639 | C | · | T | · |
| 45445432 | G | · | T | · | 45730971 | G | · | A | · |
| 45445456 | T | · | C | · | 45732895 | · | C | · | T |
| 45445479 | G | · | T | · | 45733114 | · | G | · | A |
| 45445540 | T | · | C | · | 45734129 | · | C | · | T |
| 45445563 | G | · | A | · | 45735812 | T | · | C | · |
| 45445637 | G | · | A | · | 45735976 | · | C | · | G |
| 45445696 | A | · | G | · | 45736530 | · | C | · | T |
| 45445814 | C | · | T | · | 45736710 | C | · | T | · |
| 45445936 | G | · | C | · | 45737715 | · | A | · | G |
| 45445978 | T | · | C | · | 45740350 | C | · | T | · |
| 45445995 | C | · | T | · | 45740444 | · | G | · | C |
| 45446187 | T | · | C | · | 45742824 | · | A | · | G |
| 45446497 | C | · | T | · | 45742967 | · | G | · | A |
| 45447048 | C | · | G | · | 45743080 | · | G | · | C |
| 45447827 | A | · | G | · | 45743489 | · | T | · | C |
| 45447934 | T | · | C | · | 45743510 | · | T | · | A, * |
| 45448910 | C | · | T | · | 45743512 | · | T | · | A, * |
| 45448959 | C | · | T | · | 45743668 | · | A | · | G |
| 45450842 | C | · | A | · | 45746616 | G | · | T | · |
| 45451229 | A | · | T | · | 45746854 | A | · | G | · |
| 45451809 | A | · | G | · | 45747161 | · | C | · | T |
| 45451999 | G | · | A | · | 45748744 | · | C | · | T |
| 45452116 | A | · | G | · | 45748922 | · | C | · | A |
| 45452357 | T | · | C | · | 45750160 | A | · | G | · |
| 45452577 | C | · | T | · | 45750208 | · | C | · | G |
| 45452632 | G | · | A | · | 45750746 | T | · | G | · |
| 45453178 | C | · | T, * | · | 45751547 | C | · | T | · |
| 45454444 | G | · | A | · | 45751816 | · | T | · | C |
| 45454588 | A | · | G | · | 45753724 | G | · | A | · |
| 45455558 | C | · | T | · | 45755048 | A | · | T | · |
| 45457341 | T | · | G | · | 45755428 | G | · | C | · |
| 45460307 | T | · | C | · | 45756679 | G | · | T | · |
| 45460475 | T | · | A | · | 45758723 | · | T | · | G |
| 45460579 | G | · | A | · | 45758758 | G | · | C | · |
| 45460862 | T | · | C | · | 45761833 | · | C | · | T |
| 45461152 | A | · | G | · | 45761835 | · | C | · | A |
| 45462328 | A | · | C | · | 45762011 | · | C | · | T |
| 45463027 | T | · | C | · | 45762082 | · | C | · | A |
| 45463731 | T | · | C | · | 45768804 | G | · | C | · |
| 45463950 | G | · | A | · | 45769291 | G | · | A | · |
| 45464563 | A | · | G | · | 45770335 | G | · | A | · |
| 45464844 | A | · | G | · | 45770646 | T | · | C | · |
| 45465055 | A | · | G | · | 45773473 | G | · | T | · |
| 45465362 | G | · | A | · | 45773978 | G | · | A | · |
| 45466472 | G | · | C | · | 45776795 | A | · | G | · |
| 45466908 | C | · | G | · | 45778846 | G | · | A | · |
| 45467027 | C | · | T | · | 45778894 | G | · | A | · |
| 45467262 | T | · | C | · | 45779644 | C | · | T | · |
| 45467627 | A | · | G | · | 45780643 | G | · | A | · |
| 45467911 | G | · | A | · | 45783703 | G | · | T | · |
| 45467966 | T | · | C | · | 45783796 | G | · | A | · |
| 45468001 | A | · | G | · | 45783943 | A | · | C | · |
| 45468007 | A | · | G | · | 45786197 | T | · | C | · |
| 45468060 | C | · | G | · | 45787268 | G | · | A | · |
| 45468073 | G | · | A | · | 45790549 | A | · | G | · |
| 45468096 | C | · | T | · | 45792775 | A | · | G | · |
| 45468097 | C | · | G | · | 45794439 | G | · | A | · |
| 45468099 | A | · | G | · | 45794478 | G | · | A | · |
| 45468134 | T | · | C | · | 45796559 | A | · | G | · |

POS bp position within ECA;
R1 reference allele in Icelandic horse with favorable haplotypes for the score of back and croup;
R2 reference allele in Icelandic horse with unfavorable haplotypes for the score of back and croup;
A1 alternate allele in Icelandic horse with favorable haplotypes for the score of back and croup;
A2 alternate allele in Icelandic horse with unfavorable haplotypes for the score of back and croup

### Example 3

Eleven SNP variants from ECA22 as identified in Example 1 and 2 were evaluated by MassARRAY® System (Agena Bioscience) for their association with gait performance type (trot or pace) in 178 horses of the American Standardbred breed.

A haplotype (T-G-C) including three of these SNP variants: ECA22 g.45,616,738 C>T, AX-104791028 (g.45,622,744) A>G, and AX-103683359 (g.45,662,708) T>C was strongly correlated with ability to pace. In more detail, 56 out of 89 pacing horses were homozygous or heterozygous for this haplotype (T-G-C), while the other haplotype (C-A-T) was correlated with horses that performed at the trot as 80 out of 89 trotting horses were homozygous for this haplotype, see Table 10 (Fisher Exact Test p=2.29×10⁻¹³).

TABLE 10
ECA22 haplotype associated with gait type in the Standardbred

| SNP 1 | SNP 2 | SNP 3 | | | |
|---|---|---|---|---|---|
| C | A | T | Trotter | → | 80/89 |
| T | G | C | Pacer | → | 56/89 |
| CT | AG | TC | Pacer | → | |

Hence, these SNPs can be used to analyze and predict the gait performance in terms of gait performance type trot or pace in horses.

### Example 4

Haplotype analysis was performed with the haplo.stats package v 1.8.7 in R (ref: R Core Team. R: A language and environment for statistical computing. Vienna: Foundation for Statistical Computing; 2015). The function haplo.em was used to estimate haplotypes. The haplotype effect on the tested performance trait was estimated by a generalized linear model (glm) with the function haplo.glm. Sex (stallion, mare or gelding), age, country of birth, and DMRT3 genotype were included as fixed effects. The most frequent haplotype was used as a reference and only haplotypes with frequencies greater than 0.02 were included.

The following performance traits were analyzed; number of starts, wins (number of victories), number of placings (first, second or third place), earnings and earnings per start, best km time (in seconds), gallops (number of races in which the horse was recorded to gallop) and disqualifications (number of races where the horse was disqualified as a result of galloping or pacing in the race).

The performance trait data were tested for normality by computing the skewness coefficient using the package moments v0.14. Non-normally distributed values were transformed. Log transformed values (log 10+1) were used for wins, placing, and disqualifications. Number of tarts and gallops were square root transformed. Earnings and Earnings per start were transformed applying the previously reported formula: In(Earnings+1000) (ref: Árnason T. The Importance of Different Traits in Genetic Improvement of Trotters. In: Proceedings of World Congress on Genetics Applied to Livestock Production. University of Guelph, Guelph, Canada; 1994. p. 462-70.)

The results of the haplotype analysis in 288 standardbreds are presented in Table 11.

TABLE 11
Haplotype analysis in 288 standardbreds

| SNP | SNP | SNP | SNP | SNP | SNP | SNP | SNP | | SNP | SNP | | | | p- |
|---|---|---|---|---|---|---|---|---|---|---|---|---|---|---|
| 6 | 7 | 14 | 4 | 3 | 11 | 5 | 1 | M | 2 | 12 | Trait | Coef | Freq | value |
| G | T | C | A | T | A | T | A | C | A | T | Earnings | 0.473 | 0.091 | 0.023 |
| A | C | T | C | G | G | G | G | C | A | T | Starts | −0.124 | 0.062 | 0.088 |
| G | T | C | A | T | A | T | A | C | A | T | Earnings | 0.308 | 0.091 | 0.062 |
| | | | | | | | | | | | per start | | | |
| A | C | C | C | T | G | T | A | C | A | T | Disq | 0.134 | 0.043 | 0.045 |
| A | C | T | C | G | G | G | G | C | A | T | Disq | −0.101 | 0.061 | 0.074 |
| | | | | | | | G | C | A | T | Best | −0.073 | 0.258 | 0.068 |
| | | | | | | | | | | | time | | | |
| | | | | | | | G | C | A | T | Gallops | −0.211 | 0.260 | 0.031 |
| | | | | | | | G | C | A | T | Starts | −0.064 | 0.260 | 0.091 |
| | | | | | | | A | C | A | T | Starts | 0.064 | 0.616 | 0.090 |
| | | | | | | | A | C | A | T | Gallops | 0.209 | 0.616 | 0.033 |
| | | | | | | G | G | C | A | T | Best | −0.085 | 0.153 | 0.099 |
| | | | | | | | | | | | time | | | |

SNP 1: AX-102949172, position 45,532,931 bp
SNP 2: AX-104791028, position 45,622,744 bp
SNP 3: AX-103174786, position 45,494,455 bp
SNP 4: AX-103610371, position 45,471,357 bp
SNP 5: AX-103572410, position 45,524,597 bp
SNP 6: AX-104515875, position 45,363,022 bp
SNP 7: AX-103805460, position 45,388,495 bp
SNP 11: AX-103100016, position 45,445,814 bp
SNP 12: AX-103683359, position 45,662,708 bp
SNP 14: AX-104157798, position 45,445,814 bp
M: Missense, position 45,616,738 bp

Allele A at the top SNP (SNP 1) was associated with more earnings, more earnings per start and more starts as compared to the allele G at this nucleotide position. In addition, the allele A at the top SNP was also associated with more disqualifications (disq) and more gallops. The allele G at the top position was associated with less starts, less disqualifications and less gallops but faster trotting horses. Horses that are homozygote for the allele A (AA) at the top SNP are likely to easier fall into gallop at high speed than horses that are homozygote for the allele G (GG) at the top SNP.

The missense SNP was associated with the number of starts. Horses that were homozygote for the allele T (TT) at this position had less number of starts than horses that were homozygote for the allele C (CC) or heterozygote for C (TC) at this position.

Furthermore, horses that were heterozygote for the allele T (TC) at SNP 7 were associated with more earnings as compared to horses that were homozygote for the allele C (CC) at SNP 7.

These results indicate that SNPs of the present invention are associated with performance in standardbreds.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

### REFERENCES

1. The Icelandic Agricultural Advisory Centre. General Information on Breeding Field Tests-Measurements (body measures) [Internet]. [cited 2020 Aug. 6]. Available from: https://www.rml.is/static/files/Hrossaraekt_RML/skrokkmal.pdf
2. Novoa-Bravo M, Jäderkvist Fegraeus K, Rhodin M, Strand E, Garcia L F, Lindgren G. Selection on the Colombian paso horse's gaits has produced kinematic differences partly explained by the DMRT3 gene. PLoS One. 2018 Aug. 17; 13(8):e0202584.
3. Jäderkvist K, Kangas N, Andersson L S, Lindgren G. Gaitedness is associated with the DMRT3 ‘Gait keeper’ mutation in Morgan and American Curly horses. Anim Genet. 2014 Dec. 1; 45(6):908-9.
4. Promerová M, Andersson L S, Juras R, Penedo M C T, Reissmann M, Tozaki T, et al. Worldwide frequency distribution of the ‘Gait keeper’ mutation in the DMRT3 gene. Anim Genet. 2014 Apr. 1; 45(2):274-82.
5. Velie B D, Shrestha M, Frangois L, Schurink A, Tesfayonas Y G, Stinckens A, et al. Using an Inbred Horse Breed in a High Density Genome-Wide Scan for Genetic Risk Factors of Insect Bite Hypersensitivity (IBH). Barendse W, editor. PLoS One. 2016 Apr. 12; 11(4):e0152966.
6. Finno C J, Stevens C, Young A, Affolter V, Joshi N A, Ramsay S, et al. SERPINB11 Frameshift Variant Associated with Novel Hoof Specific Phenotype in Connemara Ponies. Barsh G S, editor. PLOS Genet. 2015 Apr. 13; 11(4):e1005122.
7. Ablondi M, Viklund Å, Lindgren G, Eriksson S, Mikko S. Signatures of selection in the genome of Swedish warmblood horses selected for sport performance. BMC Genomics. 2019 Sep. 18; 20(1):717.
8. Andersson L S, Larhammar M, Memic F, Wootz H, Schwochow D, Rubin C J, et al. Mutations in DMRT3 affect locomotion in horses and spinal circuit function in mice. Nature. 2012 Aug. 30; 488(7413):642-6.
9. Fawcett J A, Sato F, Sakamoto T, Iwasaki W M, Tozaki T, Innan H. Genome-wide SNP analysis of Japanese Thoroughbred racehorses. PLoS One. 2019; 14(7).
10. Sadeghi R, Moradi-Shahrbabak M, Reza Miraei Ashtiani S, Schlamp F, Cosgrove E J, Antczak D F, et al. Genetic Diversity of Persian Arabian Horses and Their Relationship to Other Native Iranian Horse Breeds. J Hered. 2019; 173-82.
11. Velie B D, Lillie M, Fegraeus K J, Rosengren M K, Sole M, Wiklund M, et al. Exploring the genetics of trotting racing ability in horses using a unique Nordic horse model. BMC Genomics. 2019 Dec. 4; 20(1):104.
12. Velie B D, Fegraeus K J, Sole M, Rosengren M K, Roed K H, Ihler C-F, et al. A genome-wide association study for harness racing success in the Norwegian-Swedish coldblooded trotter reveals genes for learning and energy metabolism. BMC Genet. 2018; 19(1).
13. Velie B D, Lillie M, Fegraeus K J, Rosengren M K, Sole M, Wiklund M, et al. Exploring the genetics of trotting racing ability in horses using a unique Nordic horse model. BMC Genomics. 2019; 20(1).
14. Horse QTL Database [Internet]. [cited 2020 Aug. 6]. Available from: https://www.animalgenome.org/cgi-bin/QTLdb/EC/index
15. Albertsdóttir E, Eriksson S, Nssholm A, Strandberg E, Árnason T. Genetic correlations between competition traits and traits scored at breeding field-tests in Icelandic horses. Livest Sci. 2008; 114(2-3):181-7.
16. Kristjansson T, Bjornsdottir S, Albertsdóttir E, Sigurdsson A, Pourcelot P, Crevier-Denoix N, et al. Association of conformation and riding ability in Icelandic horses. Livest Sci. 2016; 189(October 2017):91-101.
17. Albertsdóttir E, Eriksson S, Sigurdsson A, Arnason T. Genetic analysis of “breeding field test status” in Icelandic horses. J Anim Breed Genet. 2011; 128(2):124-32.
18. Jönsson L. Orthopaedic health, conformation and longevity in riding horses. Doctoral thesis. SLU; 2013.
19. Liu J, Zhou Y, Liu S, Song X, Yang X Z, Fan Y, et al. The coexistence of copy number variations (CNVs) and single nucleotide polymorphisms (SNPs) at a locus can result in distorted calculations of the significance in associating SNPs to disease. Hum Genet. 2018 Jul. 1; 137(6-7):553-67.
20. JEFFCOTT L B. Disorders of the thoracolumbar spine of the horse—a survey of 443 cases. Equine Vet J. 1980 Oct. 1; 12(4):197-210.
21. Wong D, Miles K, Sponseller B. CONGENITAL SCOLIOSIS IN A QUARTER HORSE FILLY. Vet Radiol Ultrasound. 2006 May 1; 47(3):279-82.
22. Lucidi P, Bacco G, Sticco M, Mazzoleni G, Benvenuti M, Bernabo N, et al. Assessment of motor laterality in foals and young horses (Equus caballus) through an analysis of derailment at trot. Physiol Behav. 2013 Jan. 7; 109(1):8-13.
23. McGreevy P D, Thomson P C. Differences in motor laterality between breeds of performance horse. Appl Anim Behav Sci. 2006 Aug. 1; 99(1-2):183-90.
24. Cully P, Nielsen B, Lancaster B, Martin J, McGreevy P. The laterality of the gallop gait in Thoroughbred racehorses. Bartos L, editor. PLoS One. 2018 Jun. 8; 13(6):e0198545.
25. Regatieri I C, Eberth J E, Sarver F, Lear T L, Bailey E. Comparison of DMRT3 genotypes among American Saddlebred horses with reference to gait. Anim Genet. 2016 Oct. 1; 47(5):603-5.
26. Nicodemus M C, Clayton H M. Temporal variables of four-beat, stepping gaits of gaited horses. Appl Anim Behav Sci. 2003; 80(2):133-42.
27. Kim J J, Lee H I, Park T, Kim K, Lee J E, Cho N H, et al. Identification of 15 loci influencing height in a Korean population. J Hum Genet. 2010 Jan. 6; 55(1):27-31.
28. Al Abri M A, Posbergh C, Palermo K, Sutter N B, Eberth J, Hoffman G E, et al. Genome-Wide Scans Reveal a Quantitative Trait Locus for Withers Height in Horses Near the ANKRD1 Gene. J Equine Vet Sci. 2018 Jan. 1; 60:67-73.e1.
29. BLAST: Basic Local Alignment Search Tool [Internet]. [cited 2020 Aug. 7]. Available from: https://blast.ncbi.nlm.nih.gov/Blast.cgi
30. Gaudet P, Livstone M S, Lewis S E, Thomas P D. Phylogenetic-based propagation of functional annotations within the Gene Ontology consortium. Brief Bioinform. 2011; 12(5):449-62.
31. Tachmazidou I, Suveges D, Min J L, Ritchie G R S, Steinberg J, Walter K, et al. Whole-Genome Sequencing Coupled to Imputation Discovers Genetic Signals for Anthropometric Traits. Am J Hum Genet. 2017; 100(6):865-84.
32. Hnisz D, Abraham B J, Lee T I, Lau A, Saint-Andre V, Sigova A A, et al. Super-enhancers in the control of cell identity and disease. Cell. 2013 Nov. 7; 155(4):934.
33. FEIF. FEIF General Rules and Regulations [Internet]. [cited 2020 Aug. 6]. Available from: https://www.feiffengur.com/documents/FEIF_Rules_Regulations2020_complete.pdf
34. Hoyt D F, Wickler S J, Cogger E A. Time of contact and step length: the effect of limb length, running speed, load carrying and incline. J Exp Biol. 2000 Jan. 15; 203(2):221-7.
35. Sparrow L M, Pellatt E, Yu S S, Raichlen D A, Pontzer H, Rolian C. Gait changes in a line of mice artificially selected for longer limbs. PeerJ. 2017 Feb. 22; 2017 (2):e3008.
36. Pontzer H. Effective limb length and the scaling of locomotor cost in terrestrial animals. J Exp Biol. 2007 May 15; 210(10):1752-61.
37. Cook D, Gallagher P C, Bailey E. Genetics of swayback in American Saddlebred horses. Anim Genet. 2010 December; 41(SUPPL. 2):64-71.
38. WorldFengur The Studbook of Origin for the Icelandic horse [Internet]. [cited 2020 Aug. 6]. Available from: https://www.worldfengur.com/
39. Aulchenko Y S, Ripke S, Isaacs A, van Duijn C M. GenABEL: An R library for genome-wide association analysis. Bioinformatics. 2007; 23(10):1294-6.
40. R DCT. R: A Language and Environment for Statistical Computing. Foundation for Statistical Computing, Vienna Austria; 2015.
41. Rönnegård L, Shen X, Alam M. Hglm: A Package for fitting hierarchical generalized linear models. R J. 2010; 2(2):20-8.
42. Lander E, Kruglyak L. Genetic dissection of complex traits: reporting linkage results. Nat Genet. 1995; 11(november):241-7.
43. Duggal P, Gillanders E M, Holmes T N, Bailey-Wilson J E. Establishing an adjusted p-value threshold to control the family-wide type 1 error in genome wide association studies. BMC Genomics. 2008; 9:1-8.
44. Kierczak M, Jablon'ska J, Jablon'ska J, Forsberg S K G, Bianchi M, Tengvall K, et al. cgmisc: enhanced genome-wide association analyses and visualization.
45. National Center for Biotechnology Information [Internet]. [cited 2020 Aug. 6]. Available from: https://www.ncbi.nlm.nih.gov/
46. GeneCards—Human Genes | Gene Database | Gene Search [Internet]. [cited 2020 Aug. 6]. Available from: https://www.genecards.org/
47. Sambrook, J., Russell. D. W. Molecular Cloning: A Laboratory Manual, the third edition, Cold Spring Harbor Laboratory Press, Cold Spring Harbor. New York, 1.31-1.38, 2001
48. Sharma. R. C., et al. “A rapid procedure for isolation of RNA-free genomic DNA from mammalian cells”, BioTechniques, 14. 176-178. 1993.

1. A method for predicting conformation of back and croup and/or gait quality and/or gait performance type trot or pace of a horse, the method comprising determining, in a sample comprising nucleic acid molecules obtained from the horse, presence or absence of at least one biomarker useful in predicting conformation of back and croup and/or gait quality and/or gait performance type trot or pace of the horse, wherein the at least one biomarker is located in a region of from nucleotide position 44,000,000 to nucleotide position 47,000,000 on Equus caballus chromosome 22 (ECA22).

2. The method according to claim 1, further comprising predicting conformation of back and croup and/or gait quality and/or gait performance type trot or pace of the horse based on the determined presence or absence of at least one biomarker.

3. The method according to claim 1, wherein the at least one biomarker is located in a region of from nucleotide position 44,347,522 to nucleotide position 46,662,708 on ECA22.

4. The method according to claim 3, wherein the at least one biomarker is located in a region of from nucleotide position 45,347,522 to nucleotide position 46,662,708 on ECA22.

5. The method according to claim 3, wherein the at least one biomarker is located in a region of from nucleotide position 44,347,522 to nucleotide position 45,662,708 on ECA22.

6. The method according to claim 3, wherein the at least one biomarker is located in a region of from nucleotide position 45,347,522 to nucleotide position 45,662,708 on ECA22.

7. The method according to claim 6, wherein the at least one biomarker is located in a region of from nucleotide position 45,363,022 to nucleotide position 45,662,708 on ECA22.

8. The method according to claim 7, determining presence or absence of at least one biomarker comprises determining genotype of at least one single nucleotide polymorphism (SNP) selected from the group consisting of a SNP located at position 45,363,022 on ECA22, a SNP located at position 45,388,495 on ECA22, a SNP located at position 45,445,814 on ECA22, a SNP located at position 45,471,357 on ECA22, a SNP located at position 45,494,455 on ECA22, a SNP located at position 45,500,367 on ECA22, a SNP located at position 45,524,597 on ECA22, a SNP located at position 45,532,931 on ECA22, a SNP located at position 45,616,738 on ECA22, a SNP located at position 45,622,744 on ECA22, and a SNP located at position 45,662,708 on ECA22.

9. The method according to claim 8, wherein determining genotype of the at least one SNP comprises determining genotype of a SNP located at position 45,532,931 on ECA22.

10. The method according to claim 8, wherein determining genotype of the at least one SNP comprises determining genotype of at least one SNP selected from the group consisting of a SNP located at position 45,616,738 on ECA22, a SNP located at position 45,622,744 on ECA22 and a SNP located at position 45,662,708 on ECA22.

11. The method according to claim 8, wherein determining genotype of the at least one SNP comprises:
determining presence or absence of nucleotide A or G at position 45,363,022 on ECA22;
determining presence or absence of nucleotide C or T at position 45,388,495 on ECA22;
determining presence or absence of nucleotide T or C at position 45,445,814 on ECA22;
determining presence or absence of nucleotide C or A at position 45,471,357 on ECA22;
determining presence or absence of nucleotide G or T at position 45,494,455 on ECA22;
determining presence or absence of nucleotide G or A at position 45,500,367 on ECA22;
determining presence or absence of nucleotide G or T at position 45,524,597 on ECA22;
determining presence or absence of nucleotide G or A at position 45,532,931 on ECA22;
determining presence or absence of nucleotide C or T at position 45,616,738 on ECA22;
determining presence or absence of nucleotide G or A at position 45,622,744 on ECA22; and/or
determining presence or absence of nucleotide C or T at position 45,662,708 on ECA22.

12. The method according to claim 11, wherein determining genotype of the at least one SNP comprises determining presence or absence of nucleotide G or A at position 45,532,931 on ECA22.

13. The method according to claim 11, wherein determining genotype of the at least one SNP comprises:
determining presence or absence of nucleotide C or T at position 45,616,738 on ECA22;
determining presence or absence of nucleotide G or A at position 45,622,744 on ECA22; and/or
determining presence or absence of nucleotide C or T at position 45,662,708 on ECA22.

14. The method according to claim 11, wherein determining genotype of the at least one SNP comprises determining whether the horse has a first haplotype or a second haplotype, wherein
the first haplotype comprises nucleotide A at position 45,363,022 on ECA22, nucleotide C at position 45,388,495 on ECA22, nucleotide T at position 45,445,814 on ECA22, nucleotide C at position 45,471,357 on ECA22, nucleotide G at position 45,494,455 on ECA22, nucleotide G at position 45,500,367 on ECA22, nucleotide G at position 45,524,597 on ECA22, nucleotide G at position 45,532,931 on ECA22, nucleotide G at position 45,622,744 on ECA22 and nucleotide C at position 45,662,708 on ECA22; and
the second haplotype comprises nucleotide G at position 45,363,022 on ECA22, nucleotide T at position 45,388,495 on ECA22, nucleotide C at position 45,445,814 on ECA22, nucleotide A at position 45,471,357 on ECA22, nucleotide T at position 45,494,455 on ECA22, nucleotide A at position 45,500,367 on ECA22, nucleotide T at position 45,524,597 on ECA22, nucleotide A at position 45,532,931 on ECA22, nucleotide A at position 45,622,744 on ECA22 and nucleotide T at position 45,662,708 on ECA22.

15. The method according to claim 11, wherein determining genotype of the at least one SNP comprises determining whether the horse has a first haplotype or a second haplotype, wherein
the first haplotype comprises nucleotide T at position 45,616,738 on ECA22, nucleotide G at position 45,622,744 on ECA22 and nucleotide C at position 45,662,708 on ECA22; and
the second haplotype comprises nucleotide C at position 45,616,738 on ECA22, nucleotide A at position 45,622,744 on ECA22 and nucleotide T at position 45,662,708 on ECA22.

16. The method according to claim 14, wherein determining genotype of the at least one SNP comprises determining whether the horse is homozygote or heterozygote for the first haplotype or homozygote for the second haplotype.

17. A method for selection a horse for breeding, the method comprising:
determining, in a sample comprising nucleic acid molecules obtained from the horse, the allele of at least one biomarker useful in predicting conformation of back and croup and/or gait quality and/or gait performance type trot or pace of the horse, wherein the at least one biomarker is located in a region of from nucleotide position 44,000,000 to nucleotide position 47,000,00 on Equus caballus chromosome 22 (ECA22), preferably from nucleotide position 44,347,522 to nucleotide position 46,662,708 on ECA22, and more preferably from nucleotide position 45,347,522 to nucleotide position 45,662,708 on ECA22; and
selecting the horse for breeding based on the determined allele of the at least one biomarker.

18. A method for selecting a training scheme for a horse, the method comprising:
determining, in a sample comprising nucleic acid molecules obtained from the horse, the allele of at least one biomarker useful in predicting conformation of back and croup and/or gait quality and/or gait performance type trot or pace of the horse, wherein the at least one biomarker is located in a region of from nucleotide position 44,000,000 to nucleotide position 47,000,00 on Equus caballus chromosome 22 (ECA22), preferably from nucleotide position 44,347,522 to nucleotide position 46,662,708 on ECA22, and more preferably from nucleotide position 45,347,522 to nucleotide position 45,662,708 on ECA22; and
selecting the training scheme for the horse based on the determined allele of the at least one biomarker.

19. The method according to claim 18, wherein
determining the allele of the at least one biomarker comprises determining, in the sample, the allele of at least one biomarker selected from the group consisting of a single nucleotide polymorphism (SNP) located at position 45,616,738 on ECA22, a SNP located at position 45,622,744 on ECA22 and a SNP located at position 45,662,708 on ECA22; and
selecting the training scheme for the horse comprises selecting a training scheme adapted for pacers or a training scheme adapted for trotters based on the determined allele of the at least one biomarker.

20. The method according to claim 19, wherein
determining the allele of the at least one biomarker comprises determining, in the sample, whether the horse has a first haplotype or a second haplotype, wherein
the first haplotype comprises nucleotide T at position 45,616,738 on ECA22, nucleotide G at position 45,622,744 on ECA22 and nucleotide C at position 45,662,708 on ECA22; and
the second haplotype comprises nucleotide C at position 45,616,738 on ECA22, nucleotide A at position 45,622,744 on ECA22 and nucleotide T at position 45,662,708 on ECA22; and
selecting the training scheme for the horse comprises:
selecting the training scheme adapted for pacers if the horse is homozygous or heterozygous for the first haplotype; and
selecting the training scheme adapted for trotters if the horse is homozygous for the second haplotype.

21. A kit for predicting conformation of back and croup and/or gait quality and/or gait performance type trot or pace of a horse, the kit comprises:
at least one oligonucleotide probe capable of forming a hybridized nucleic acid with a single nucleotide polymorphism (SNP) or a nucleic acid region flanking the SNP, wherein the SNP is selected from the group consisting of a SNP located at position 45,363,022 on Equus caballus chromosome 22 (ECA22), a SNP located at position 45,388,495 on ECA22, a SNP located at position 45,445,814 on ECA22, a SNP located at position 45,471,357 on ECA22, a SNP located at position 45,494,455 on ECA22, a SNP located at position 45,500,367 on ECA22, a SNP located at position 45,524,597 on ECA22, a SNP located at position 45,532,931 on ECA22, a SNP located at position 45,622,744 on ECA22, and a SNP located at position 45,662,708 on ECA22; and
instructions for predicting conformation of back and croup and/or gait quality and/or gait performance type in the horse based on the horse's genotype at the SNP.

22. A kit for predicting conformation of back and croup and/or gait quality and/or gait performance type trot or pace of a horse, the kit comprises:
at least one oligonucleotide probe capable of forming a hybridized nucleic acid with a single nucleotide polymorphism (SNP) or a nucleic acid region flanking the SNP, wherein the SNP is selected from the group consisting of a SNP located at position 45,616,738 on Equus caballus chromosome 22 (ECA22), a SNP located at position 45,622,744 on ECA22, and a SNP located at position 45,662,708 on ECA22; and
instructions for predicting conformation of back and croup and/or gait quality and/or gait performance type in the horse based on the horse's genotype at the SNP.
